(19) United States
(12) Patent Application Publication
(10) Pub. No.: US 2010/0063071 A1
(43) Pub. Date: Mar. 11, 2010

(54) THERAPEUTIC COMPOSITIONS AND RELATED METHODS OF USE

(76) Inventors: William F. Kiesman, Wayland, MA (US); Mary Fure, East Hampstead, NH (US); John Kuczek, Lowell, MA (US); Aaron Deykin, Needham, MA (US); Barry Ticho, Cambridge, MA (US); David Bird, San Diego, CA (US); Vince Covari, Carmel, IN (US)

Correspondence Address: LANDO & ANASTASI, LLP;B2047, ONE MAIN STREET, SUITE 1100, CAMBRIDGE, MA, 02142, US

(21) Appl. No.: 12/483,836

(22) Filed: Jun. 12, 2009

Related U.S. Application Data
(60) Provisional application No. 61/061,510, filed on Jun. 13, 2008.
(60) Provisional application No. 61/098,519, filed on Sep. 19, 2008.

(51) Int. Cl. A61K 31/52; A61P 9/00

(52) U.S. Cl. 514/263.24

(57) ABSTRACT

Compositions and dosage forms including adenosine receptor antagonists such as 3-[4-(2,6-Dioxo-1,3-dipropyl-2,3,6,7-tetrahydro-1H-purin-8-yl)-bicyclo[2.2.2]oct-1-yl]-propionic acid and methods of using the same are described herein.

### PRIORITY CLAIM

The present application claims the benefit of U.S. provisional application No. 61/061,510, filed Jun. 13, 2008 and U.S. provisional application No. 61/098,519, filed Sep. 19, 2008, the contents of each of which are incorporated herein by reference.

### FIELD OF THE INVENTION

This invention relates to adenosine receptor antagonists and methods of use thereof.

### BACKGROUND OF INVENTION

Adenosine is a ubiquitous biochemical messenger. Adenosine binds to and activates seven-transmembrane spanning G-protein coupled receptors, eliciting a variety of physiological responses. Adenosine receptors are divided into four known subtypes (i.e., A₁, A₂ₐ, A_{2b}, and A₃). These receptor subtypes mediate different, and sometimes opposing, effects. Activation of the adenosine A₁ receptor, for example, elicits an increase in renal vascular resistance, while activation of the adenosine A₂ₐ receptor elicits a decrease in renal vascular resistance.

In most mammalian organ systems, periods of metabolic stress result in significant increases in the concentration of adenosine in the tissue. The heart, for instance, produces and releases adenosine to mediate adaptive responses to stress, such as reductions in heart rate and coronary vasodilatation. Likewise, adenosine concentrations in kidneys increase in response to hypoxia, metabolic stress and many nephrotoxic substances. The kidneys also produce adenosine constitutively. The kidneys adjust the amount of constitutively produced adenosine in order to regulate glomerular filtration and electrolyte reabsorption. Regarding control of glomerular filtration, activation of A₁ receptors leads to constriction of afferent arterioles, while activation of A₂ₐ receptors leads to dilatation of efferent arterioles. Activation of A₂ₐ receptors exerts vasodilatory effects on the afferent arteriole. Overall, the effect of activation of these glomerular adenosine receptors is to reduce glomerular filtration rate. In addition, A₁ adenosine receptors are located in the proximal tubule and distal tubular sites. Activation of these receptors stimulates sodium reabsorption from the tubular lumen. Accordingly, blocking the effects of adenosine on these receptors produces a rise in glomerular filtration rate and an increase in sodium excretion.

### SUMMARY OF INVENTION

Applicants have discovered that certain compositions, dosage forms and methods of administration of adenosine receptor antagonists can be both safe and efficacious for the subject, for example, in the treatment of acute decompensated heart failure or other disorders in which treatment with an adenosine receptor antagonist would be beneficial (e.g., metabolic syndrome).

In one aspect, the invention features a method of administering an adenosine receptor antagonist. The method includes administering to a subject (for example, a subject in need thereof) a liquid formulation comprising the adenosine receptor antagonist by infusing the liquid formulation into the subject, wherein the amount of adenosine receptor antagonist administered to the subject is from about 0.03 mg/kg to about 3.0 mg/kg.

In some embodiments, the method also includes reconstituting a solid formulation including the adenosine receptor antagonist to produce the liquid formulation, for example, a solution.

In some embodiments, the amount of adenosine receptor antagonist administered to the subject is from about 0.03 mg/kg to about 1.0 mg/kg, for example, from about 0.03 mg/kg to about 0.3 mg/kg, about 0.03 mg/kg, about 0.15 mg/kg, or about 0.3 mg/kg.

In some embodiments, the adenosine receptor antagonist is administered by infusion. In some embodiments, the adenosine receptor antagonist is infused over a course of about 30 minutes, e.g., from about 15 to about 45 minutes, from about 20 to about 40 minutes, from about 25 to about 35 minutes.

In some embodiments, administering the adenosine receptor antagonist promotes natriuresis, reduces body weight, and/or preserves renal function.

In some embodiments, the adenosine receptor antagonist is administered two times daily (e.g., about every 12 hours). In some embodiments, the adenosine receptor antagonist is administered to the subject two times daily for at least 1 to 21 days, for example about 5 days. In some embodiments, the adenosine receptor antagonist is administered once daily. In some embodiments, the adenosine receptor antagonist is administered to the subject once daily for at least 1 to 21 days, for example about 5 days. In some embodiments, the total daily dose of the adenosine receptor antagonist is from about 6 mg to about 50 mg.

In some embodiments, the liquid formulation further includes histidine. In some embodiments, the pH of the liquid formulation is at least about 8.0 e.g., from about 8.0 to about 10.0 or from about 8.5 to about 9.5 e.g., about 8.5 or about 9.5. In some embodiments, the liquid formulation further includes a base, for example, NaOH.

In some embodiments, the liquid formulation further includes a pharmaceutically acceptable carrier, for example, mannitol.

In some embodiments, the adenosine receptor antagonist is a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid.

In some embodiments, the adenosine receptor antagonist is administered in combination with an additional therapeutic agent, for example, an anti-seizure medication such as cabamazepine, clobazam, diastat, ethosuximide, felbamate, frisium, gabapentin, klonopin, lamotrigine, levetiracetam, lorazepam, luminal, lyrica, oxcarbazepine, phenobarbital, phenytoin, primidone, rufinamide, sabril, tiagabine, topiramate, valproic acid, valproate, or zonisamide. In some embodiments, the additional therapeutic agent is a diuretic, for example, a loop diuretic such as furosemide, bumetanide, ethacrynic adic, or orsemide.

In some embodiments, an ischemic event is imminent in the subject, for example, an ischemic event such as acute coronary syndrome, stroke, organ transplantation (e.g., organ transplantation surgery), kidney ischemia, or shock. In some embodiments, the adenosine receptor antagonist is administered is administered within two days before or after an ischemic event (e.g., two days after).

In some embodiments, the subject is suffering from acute decompensated heart failure. Adenosine receptor antagonist is administered the subject is also suffering from concomitant renal insufficiency.

In some embodiments, the subject is at low risk for seizures. For example, the subject does not have a history of past seizures (currently, no stroke or head trauma within the past 6 months, no seizure within the last 10 years, and subject has not been on any anti-seizure medication for the past 5 years).

In some embodiments, the subject has a GFR of from about 10 to about 80 (e.g., from about 20 to about 70).

In one aspect, the invention features a course of administering an adenosine receptor antagonist to a subject (for example, a subject in need thereof). The course includes intravenously administering to the subject a liquid formulation comprising the adenosine receptor antagonist; wherein the adenosine receptor antagonist is administered intravenously once or twice daily to the subject for at least 2 days; and upon completion of the course of intravenous administration of the adenosine receptor antagonist, administering to the subject orally an adenosine receptor antagonist, for at least 1 day.

In some embodiments, the amount of adenosine receptor antagonist administered intravenously to the subject is from about 0.03 mg/kg to about 3.0 mg/kg, about 0.03 mg/kg to about 1.0 mg/kg, for example, from about 0.03 mg/kg to about 0.3 mg/kg, about 0.03 mg/kg, about 0.15 mg/kg, or about 0.3 mg/kg per intravenous administration.

In some embodiments, the subject is administered orally the adenosine receptor antagonist for at least about 7 days.

In some embodiments, the adenosine receptor antagonist is administered by infusion. In some embodiments, the adenosine receptor antagonist is infused over a course of about 30 minutes, e.g., from about 15 to about 45 minutes, from about 20 to about 40 minutes, from about 25 to about 35 minutes.

In some embodiments, administering the adenosine receptor antagonist promotes natriuresis, reduces body weight, and/or preserves renal function.

In some embodiments, the adenosine receptor antagonist is administered two times daily (e.g., about every 12 hours).

In some embodiments, the adenosine receptor antagonist is administered intravenously to the subject two times daily for at least 1 to 21 days, for example about 5 days. In some embodiments, the adenosine receptor antagonist is administered intravenously once daily. In some embodiments, the adenosine receptor antagonist is administered intravenously to the subject once daily for at least 1 to 21 days, for example about 5 days. In some embodiments, the total daily dose of the adenosine receptor antagonist is from about 6 mg to about 50 mg. In some embodiments, the adenosine receptor antagonist is administered intravenously at which time the subject is hospitalized.

In some embodiments, the liquid formulation further includes histidine. In some embodiments, the pH of the liquid formulation is at least about 8.0 e.g., from about 8.0 to about 10.0 or from about 8.5 to about 9.5 e.g., about 8.5 or about 9.5. In some embodiments, the liquid formulation further includes a base, for example, NaOH.

In some embodiments, the liquid formulation further includes a pharmaceutically acceptable carrier, for example, mannitol.

In some embodiments, the adenosine receptor antagonist is a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid.

In some embodiments, the subject is suffering from acute decompensated heart failure. Adenosine receptor antagonist is administered the subject is also suffering from concomitant renal insufficiency.

In one aspect, the invention features a method of selecting a patient for treatment with an adenosine receptor antagonist (e.g., a patient in need thereof). The method includes identifying a patient that is not at an elevated risk of seizure, thereby selecting a patient for treatment with an adenosine receptor antagonist.

In some embodiments, the method also includes administering to the subject an adenosine receptor antagonist (e.g., administering 3-[4-(2,6-Dioxo-1,3-dipropyl-2,3,6,7-tetrahydro-1H-purin-8-yl)-bicyclo[2.2.2]oct-1-yl]-propionic acid). The adenosine receptor antagonist can be administered using any of the methods or courses of administration described herein. For example, the adenosine receptor antagonist can be administered by infusion, e.g., over a course of about 30 minutes, and can be administered once or twice daily.

In one aspect, the invention features a pharmaceutical preparation. The preparation includes an adenosine receptor antagonist (e.g., 3-[4-(2,6-Dioxo-1,3-dipropyl-2,3,6,7-tetrahydro-1H-purin-8-yl)-bicyclo[2.2.2]oct-1-yl]-propionic acid) and histidine.

In some embodiments, the preparation includes a molar ratio of adenosine receptor antagonist:histidine of from about 5:1 to about 0.5:1 e.g., from about 3:1 to about 1:1, for example, about 2.5:1, about 2:1, or about 1.5:1.

In some embodiments, the preparation also includes a base, for example, NaOH.

In some embodiments, the preparation further includes a pharmaceutically acceptable carrier, for example, mannitol.

In some embodiments, the preparation is a solid, e.g., a particulate solid and/or a lyophilite. In some embodiments, the preparation is a liquid, for example, a liquid including water such as an aqueous solution. In some embodiments, when the preparation is aqueous solution, the pH of the preparation is at least about 8.0 e.g., from about 8.0 to about 10.0 or from about 8.5 to about 9.5 e.g., about 8.5 or about 9.5.

In one aspect, the invention features a dosage form of an adenosine receptor antagonist. The dosage form includes from about 1 to about 75 mg of the adenosine receptor antagonist. In some embodiments, the dosage form includes about 2 mg of the adenosine receptor antagonist. In some embodiments, the dosage form includes about 10 mg of the adenosine receptor antagonist. In some embodiments, the dosage form includes about 20 mg of the adenosine receptor antagonist. In some embodiments, the dosage form includes about 60 mg of the adenosine receptor antagonist.

In some embodiments, the dosage form is for administration by infusion.

In some embodiments, the dosage form also includes histidine, for example, from about 0.2 mg to about 15 mg histidine, e.g., about 0.4 mg, about 2 mg, about 4 mg, or about 12 mg. In some embodiments, the dosage form includes a molar ratio of adenosine receptor antagonist:histidine of from about 5:1 to about 0.5:1 e.g., from about 3:1 to about 1:1, for example, about 2.5:1, about 2:1, or about 1.5:1.

In some embodiments, the dosage form is a solid, e.g., a particulate solid and/or a lyophilite. In some embodiments, the dosage form is a liquid, for example, a liquid including water such as an aqueous solution. In some embodiments, when the dosage form is aqueous solution, the pH of the preparation is at least about 8.0 e.g., from about 8.0 to about 10.0 or from about 8.5 to about 9.5 e.g., about 8.5 or about 9.5. In some embodiments, the concentration of the adenosine receptor antagonist is from about 0.05 mM about 10 mM (e.g., is from about 0.1 mM to about 5 mM, about 0.5 mM or about 1 mM). In some embodiments, the dosage form includes from about 25 ml to about 100 ml water (e.g., about 50 ml).

In one aspect, the invention features a method of preparing a liquid dosage form. The method includes supplying a solid dosage form comprising an adenosine receptor antagonist and histidine; and combining the solid dosage form with a liquid to provide a liquid dosage form.

In some embodiments, the dosage form includes about 2 mg of the adenosine receptor antagonist. In some embodiments, the dosage form includes about 10 mg of the adenosine receptor antagonist. In some embodiments, the dosage form includes about 20 mg of the adenosine receptor antagonist. In some embodiments, the dosage form includes about 60 mg of the adenosine receptor antagonist.

In some embodiments, the dosage form also includes histidine, for example, from about 0.2 mg to about 15 mg histidine, e.g., about 0.4 mg, about 2 mg, about 4 mg, or about 12 mg. In some embodiments, the dosage form includes a molar ratio of adenosine receptor antagonist:histidine of from about 5:1 to about 0.5:1 e.g., from about 3:1 to about 1:1, for example, about 2.5:1, about 2:1, or about 1.5:1.

In some embodiments, the liquid is water. In some embodiments, the liquid dosage form is an aqueous solution, for example, having a pH of at least about 8.0 e.g., from about 8.0 to about 10.0 or from about 8.5 to about 9.5 e.g., about 8.5 or about 9.5.

In some embodiments, the concentration of the adenosine receptor antagonist is from about 0.05 mM about 10 mM (e.g., is from about 0.1 mM to about 5 mM, about 0.5 mM, or about 1 mM).

In one aspect, the invention features a method of providing instructions for preparing a liquid dosage form. The method includes instructing the preparer to obtain solid dosage form of an adenosine receptor antagonist, and of dry and combine the solid dosage form with a liquid to provide the liquid dosage, thereby instructing.

In some embodiments, the solid dosage form is a lyophilite.

In some embodiments, liquid dosage form includes a concentration of the adenosine receptor antagonist of from about 0.05 mM about 10 mM (e.g., is from about 0.1 mM to about 5 mM, about 0.5 mM or about 1 mM).

In some embodiments, the solid dosage form also includes histidine, for example, from about 0.2 mg to about 15 mg histidine, e.g., about 0.4 mg, about 2 mg, about 4 mg, or about 12 mg. In some embodiments, the solid dosage form includes a molar ratio of adenosine receptor antagonist:histidine of from about 5:1 to about 0.5:1 e.g., from about 3:1 to about 1:1, for example, about 2.5:1, about 2:1, or about 1.5:1.

In one aspect, the invention features a diluent transfer system. The system includes:

a vial containing a predetermined volume of a drug to be delivered to a patient; and

a syringe containing a volume of a diluent, the volume of the diluent and the predetermined volume of the drug is in a ratio of 1.1:1.

In some embodiments, the transfer also includes an intravenous bag containing a carrier liquid.

In some embodiments, the transfer also includes instructions for at least one of dosing and delivery of the drug to the patient.

In some embodiments, the predetermined volume of the drug is at least 3 mL.

In some embodiments, the instructions include dosing guidelines based at least partially on a weight of the patient.

In some embodiments, the predetermined volume corresponds to 20 mg of the diluent.

In one aspect, the invention features a drug delivery system. The drug delivery system includes:

a dual chamber syringe having a first chamber containing a drug to be delivered to a patient and a second chamber containing a diluent, wherein a ratio of a volume of the drug and a volume of the diluent is 1:1.

In some embodiments, the drug delivery system also includes an intravenous carrier system.

In some embodiments, the drug delivery system also includes instructions for at least one of dosing and delivery of the drug to the patient.

In one aspect, the invention features a drug delivery system. The drug delivery system includes:

a vial containing a predetermined volume of a “reconstituted” (or “stabilized”) drug; and

an intravenous bag containing a carrier.

In some embodiments, the drug delivery system also includes instructions for at least one of dosing and delivery of the drug to the patient.

In some embodiments, the vial includes an outlet port, a first seal disposed over the outlet port and wherein the intravenous bag comprising an inlet port sized to receive the outlet port of the vial.

In some embodiments, the drug delivery system also includes a syringe sized to contain a volume of the drug.

In some embodiments, the instructions include dosing guidelines that provide incremental dosages of the drug based at least partially on the weight of the patient.

In one aspect, the invention features a method of intravenous drug delivery. The method includes:

providing a drug in a plurality of vials, each of the plurality of vials having a predetermined volume;

providing a syringe containing a volume of a diluent, the volume of the diluent and the predetermined volume of the drug is in a predefined ratio; and

providing instructions for at least one of dosing and delivery of the drug to a patient.

In some embodiments, the method also includes providing an intravenous bag containing a carrier liquid and wherein the instructions comprises dosing guidelines based at least partially on a weight of the patient.

In some embodiments, the method also includes determining a number of required vials to provide a desired dose to be delivered based at least partially on the weight of the patient. In some embodiments, the predefined ratio is 1.1:1.

In one aspect, the invention features, a method of monitoring a subject, the method comprising administering to a subject an adenosine receptor antagonist, the method comprising administering to a subject a liquid formulation comprising the adenosine receptor antagonist by infusing the liquid formulation into the subject, wherein the amount of adenosine receptor antagonist administered to the subject is from about 0.03 mg/kg to about 3.0 mg/kg; and monitoring the subject.

In some embodiments, the patient is monitored for an adverse event or improvement in condition. In some embodiments, the monitoring comprises assessing one or more pharmacodynamic parameters. In some embodiments, the pharmacodynamic parameter is selected from the group consisting of sodium excretion, urine volume, adjusted creatinine clearance, hemodynamics, and body weight. In some embodiments, the subject is monitored for an adverse event such as a seizure. In some embodiments, the adverse event is status epilepticus. In some embodiments, the adenosine receptor antagonist is a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid.

In one aspect, the invention features a method of monitoring a subject, the method comprising: administering to a subject, 3-[4-(2,6-Dioxo-1,3-dipropyl-2,3,6,7-tetrahydro-1H-purin-8-yl)-bicyclo[2.2.2]oct-1-yl]-propionic acid; and monitoring the subject, for example, for an adverse event or improvement in condition.

In some embodiments, the monitoring comprises assessing one or more pharmacodynamic parameters. In some embodiments, the pharmacodynamic parameter is selected from the group consisting of sodium excretion, urine volume, adjusted creatinine clearance, hemodynamics, and body weight. In some embodiments, the subject is monitored for an adverse event such as a seizure. In some embodiments, wherein the adverse event is status epilepticus.

### BRIEF DESCRIPTION OF DRAWINGS

FIG. 1 depicts exemplary adenosine receptor antagonists.

FIG. 2 depicts a kit for preparing a single dosage form of an adenosine receptor antagonist including a syringe, a bag, and a bottle.

FIG. 3 depicts a kit for preparing a single dosage form of an adenosine receptor antagonist including a multi-chamber syringe.

FIG. 4 depicts a kit including a single dosage form of an adenosine receptor antagonist, wherein the kit includes a cap portion and a bag portion, wherein the cap portion and the bag portion are separated by a membrane.

FIG. 5 depicts a kit for preparing a single dosage form of an adenosine receptor antagonist including a syringe, a bag, and a bottle.

FIG. 6 depicts a kit for preparing a single dosage form of an adenosine receptor antagonist including a syringe and a bottle.

### DETAILED DESCRIPTION

Compositions of adenosine receptor antagonists, dosage forms of adenosine receptor antagonists, and methods of administering adenosine receptor antagonists, are described herein.

Adenosine Receptor Antagonists

The compositions, dosage forms, and methods described herein can be used with any known adenosine receptor antagonists. As used herein, an “antagonist” is a molecule that binds to a receptor without activating the receptor. It competes with the endogenous ligand for this binding site and, thus, reduces the ability of the endogenous ligand to stimulate the receptor. In the context of the present invention, a “selective antagonist” is an antagonist that binds to a specific subtype of adenosine receptor with higher affinity than to other adenosine receptor subtypes. The antagonists of the invention can, for example, have high affinity for receptors and are selective, having (a) nanomolar binding affinity for the receptor and (b) at least 10 times, e.g., 50 times, e.g., at least 100 times, greater affinity for the receptor subtype than for any other receptor subtype.

Exemplary adenosine receptor antagonists include the following.

Exemplary adenosine receptor antagonists include compounds of formula (I):

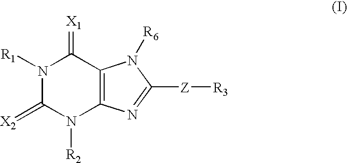

where R₁ and R₂ are independently chosen from: (a) hydrogen; (b) alkyl, alkenyl of not less than 3 carbons, or alkynyl of not less than 3 carbons; wherein the alkyl, alkenyl, or alkynyl is either unsubstituted or functionalized with one or two substituents selected from the group consisting of hydroxy, alkoxy, amino, alkylamino, dialkylamino, heterocyclyl, acylamino, alkylsulfonylamino, and heterocyclylcarbonylamino; and (c) aryl and substituted aryl.

R₃ is a bicyclic or tricyclic group chosen from:

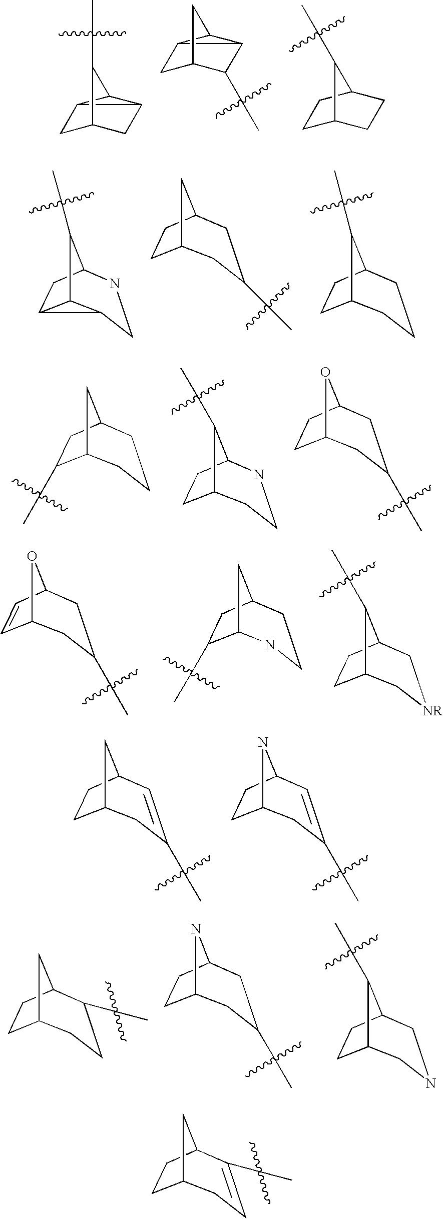

where the bicyclic or tricyclic group can be unsubstituted or can be functionalized with one or more (e.g., one, two, three, or more) substituents chosen from: (a) alkyl, alkenyl, and alkynyl; wherein the alkyl, alkenyl, and alkynyl are either unsubstituted or functionalized with one or more substituents selected from the group consisting of alkoxy, alkoxycarbonyl, alkoxycarbonylaminoalkylamino, aralkoxycarbonyl, —R5, dialkylamino, heterocyclylalkylamino, hydroxy, substituted arylsulfonylaminoalkylamino, and substituted heterocyclylaminoalkylamino; (b) acylaminoalkylamino, alkenylamino, alkoxycarbonyl, alkoxycarbonyl, alkoxycarbonylalkylamino, alkoxycarbonylaminoacyloxy, alkoxycarbonylaminoalkylamino, alkylamino, amino, aminoacyloxy, carbonyl, —R₅, R₅-alkoxy, R₅-alkylamino, dialkylaminoalkylamino, heterocyclyl, heterocyclylalkylamino, hydroxy, phosphate, substituted arylsulfonylaminoalkylamino, substituted heterocyclyl, and substituted heterocyclylaminoalkylamino.

R₄ is chosen from —H, —C₁₋₄-alkyl, —C₁₋₄-alkyl-CO₂H, and phenyl; and can be unsubstituted or can be functionalized with one or more substituents chosen from halogen, —OH, —OMe, —NH₂, —NO₂ and benzyl, optionally substituted with one, two, or three groups selected from halogen, —OH, —OMe, —NH₂, and —NO₂.

R₅ is chosen from —CH₂COOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, and the following:

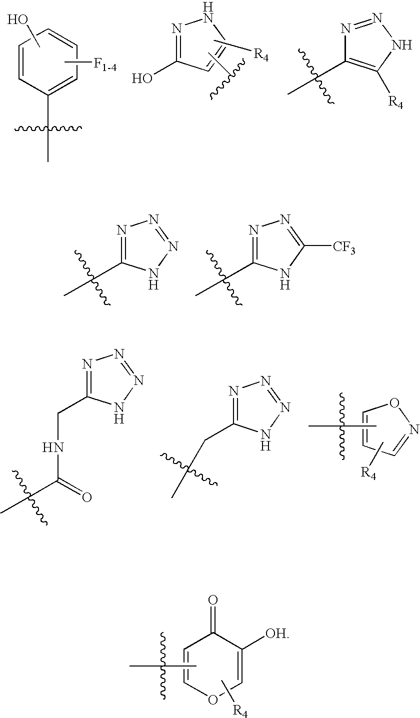

X₁ and X₂ are chosen, independently, from oxygen (O) and sulfur (S).

Z is chosen from a single bond, —O—, —(CH₂)₁₋₃—, —O(CH₂)₁₋₂—, CH₂OCH₂—, —(CH₂)₁₋₂O—, —CH═CHCH₂—, —CH═CH—, and —CH₂CH═CH—.

R₆ is chosen from hydrogen, alkyl, acyl, alkylsulfonyl, aralkyl, substituted aralkyl, substituted alkyl, and heterocyclyl.

In some embodiments, R₆ is hydrogen. However, when R₆ is methyl or another non-hydrogen substituent, the compounds can be highly selective for inhibition of adenosine A₂ₐ receptors.

In certain embodiments, R₁ and R₂ can be the same or different alkyl groups. For example, one or both can be n-propyl.

In some embodiments, Z is a single bond.

In one embodiment, R₃ is chosen from the following bicyclic and tricyclic structures:

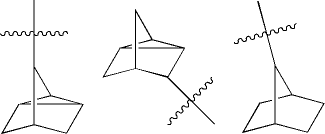

and is functionalized with one or more substituents chosen from carbonyl, hydroxy, alkenyl, alkenyloxy, hydroxyalkyl, carboxy, carboxyalkenyl, carboxyalkyl, aminoacyloxy, carboxyalkoxy, dialkylaminoalkenyl, and dialkylaminoalkyl.

In another embodiment, R₃ is:

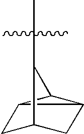

and is functionalized with one or more substituents chosen from carbonyl, hydroxy, alkenyl, carboxyalkenyl, hydroxyalkyl, dialkylaminoalkenyl, and dialkylaminoalkyl. Thus, for example, the compound can be 8-(5-Hydroxy-tricyclo[2.2.1.0^{2,6}]hept-3-yl)-1,3-dipropyl-3,7-dihydro-purine-2,6-dione; 8-(5-Hydroxymethyl-tricyclo[2.2.1.0^{2,6}]hept-3-yl)-1,3-dipropyl-3,7-dihydro-purine-2,6-dione; 8-[5-(3-Dimethylaminopropylidene)-tricyclo[2.2.1.0^{2,6}]hept-3-yl]-1,3-dipropyl-3,7-dihydro-purine-2,6-dione; or 8-[5-(3-Dimethylaminopropyl)-tricyclo[2.2.1.0^{2,6}]hept-3-yl]-1,3-dipropyl-3,7-dihydro-purine-2,6-dione.

In still another embodiment, R₃ is:

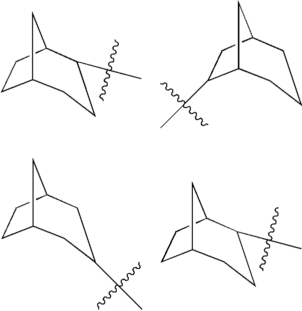

and is functionalized with one or more substituents chosen from hydroxy, carbonyl, alkyl, —R₅, R₅-alkyl, dialkylaminoalkylamino, alkoxycarbonylalkylamino, R₅-alkylamino, heterocyclyl, alkenylamino, amino, alkylamino, heterocyclylalkylamino, acylaminoalkylamino, phosphate, heterocyclylaminoalkylamino, and heterocyclylaminoalkylaminoalkyl.

In yet another embodiment, R₃ is:

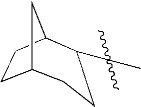

and is functionalized with one or more substituents chosen from hydroxy, —R₅, R₅-alkyl, and hydroxyalkyl. Thus, for example, the compound can be 4-(2,6-Dioxo-1,3-dipropyl-2,3,6,7-tetrahydro-1H-purin-8-yl)-bicyclo[3.2.1]octane-1-carboxylic acid.

In another embodiment, R₃ is:

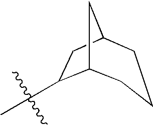

and is functionalized with one or more substituents chosen from alkyl, hydroxy, carbonyl, —R₅, and R₅-alkyl. Thus, for example, the compound can be 8-(4-Hydroxy-bicyclo[3.2.1]oct-6-yl)-1,3-dipropyl-3,7-dihydro-purine-2,6-dione; or 8-(4-Oxo-bicyclo[3.2.1]oct-6-yl)-1,3-dipropyl-3,7-dihydro-purine-2,6-dione.

In still another embodiment, R₃ is:

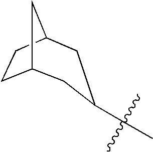

and is functionalized with one or more substituents chosen from carbonyl, hydroxy, dialkylaminoalkylamino, —R⁵, and substituted heterocyclylaminoalkylaminoalkyl. Thus, for example, the compound can be 8-[8-(2-Dimethylaminoethylamino)-bicyclo[3.2.1]oct-3-yl]-1,3-dipropyl-3,7-dihydro-purine-2,6-dione; or 8-(8-Hydroxy-bicyclo[3.2.1]oct-3-yl)-1,3-dipropyl-3,7-dihydro-purine-2,6-dione.

In yet another embodiment, R₃ is:

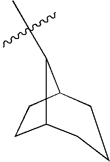

and is functionalized with one or more substituents chosen from carbonyl, hydroxy, and —R5. Thus, for example, the compound can be 8-(3-Hydroxy-bicyclo[3.2.1]oct-8-yl)-1,3-dipropyl-3,7-dihydro-purine-2,6-dione.

In yet another embodiment, R₃ is selected from bicycles:

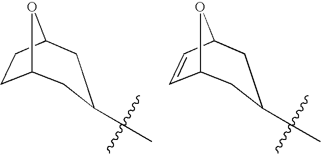

and is functionalized with one or more substituents chosen from hydroxyalkyl, hydroxy, and alkoxycarbonyl. Thus, for example, the compound can be 8-(8-Oxa-bicyclo[3.2.1]oct-6-en-3-yl)-1,3-dipropyl-3,7-dihydro-purine-2,6-dione.

In yet another embodiment, R₃ is:

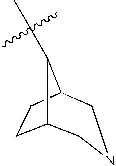

and is functionalized with one or more substituents chosen from carbonyl, aralkyloxycarbonylalkyl, and alkoxycarbonylalkyl. Thus, for example, the compound can be 8-(2-Oxo-3-aza-bicyclo[3.2.1]oct-8-yl)-1,3-dipropyl-3,7-dihydro-purine-2,6-dione.

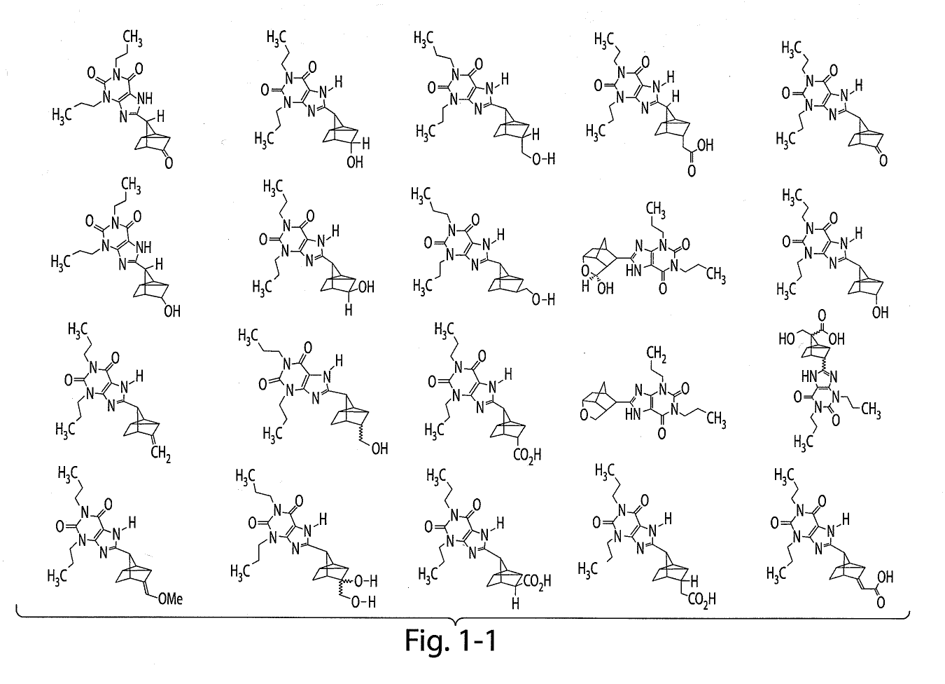

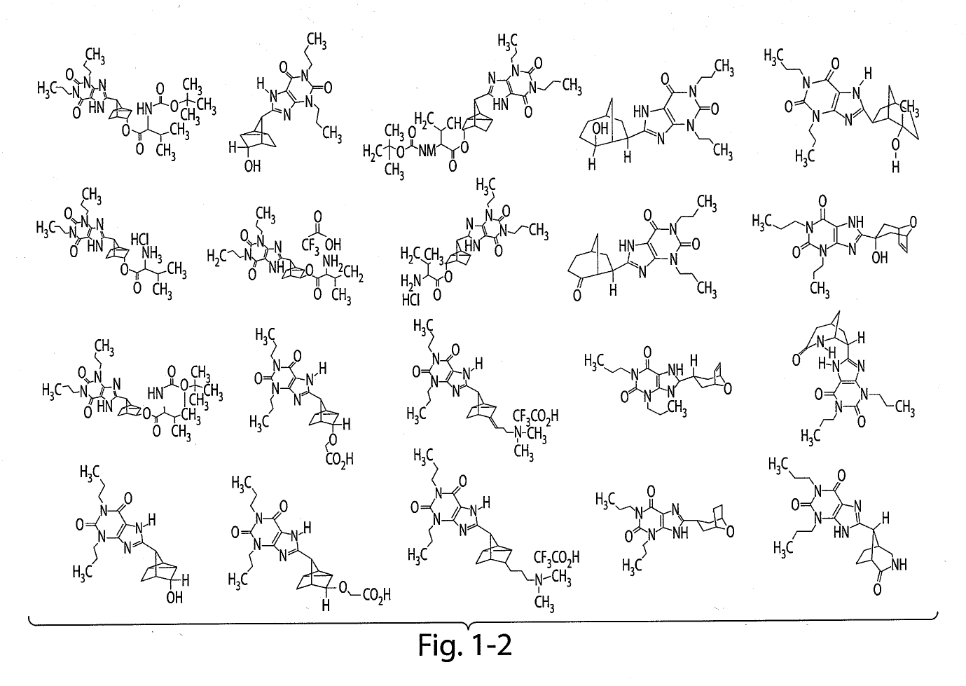

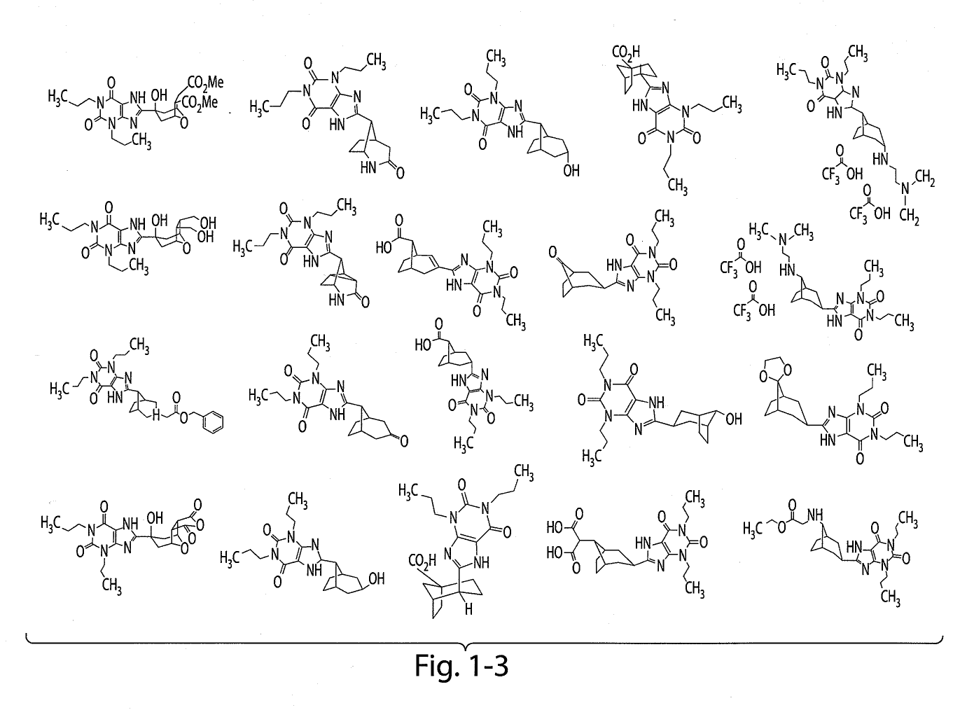

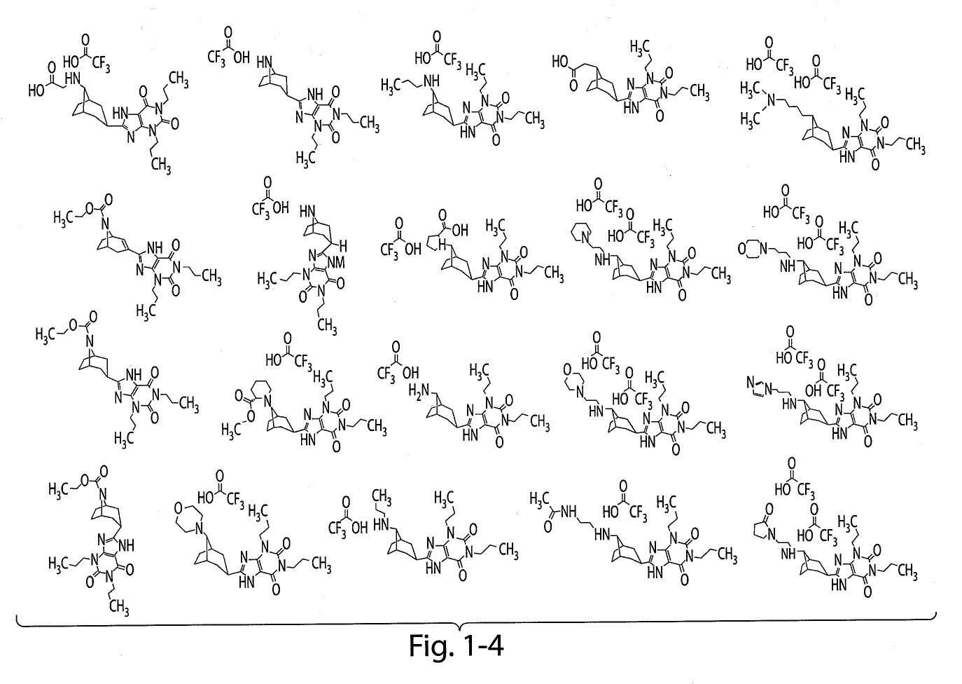

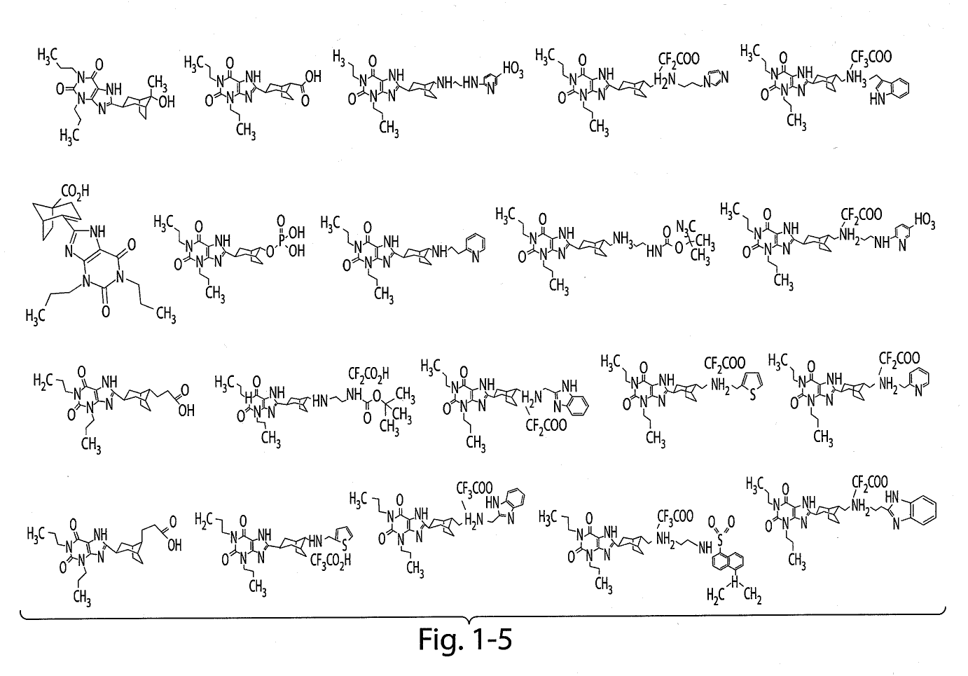

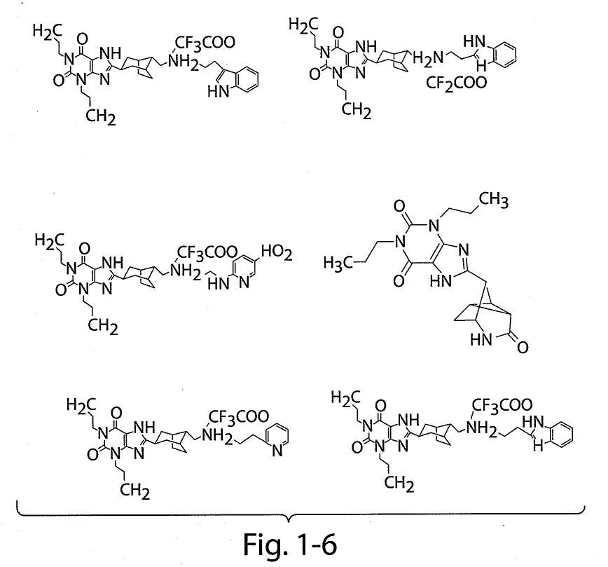

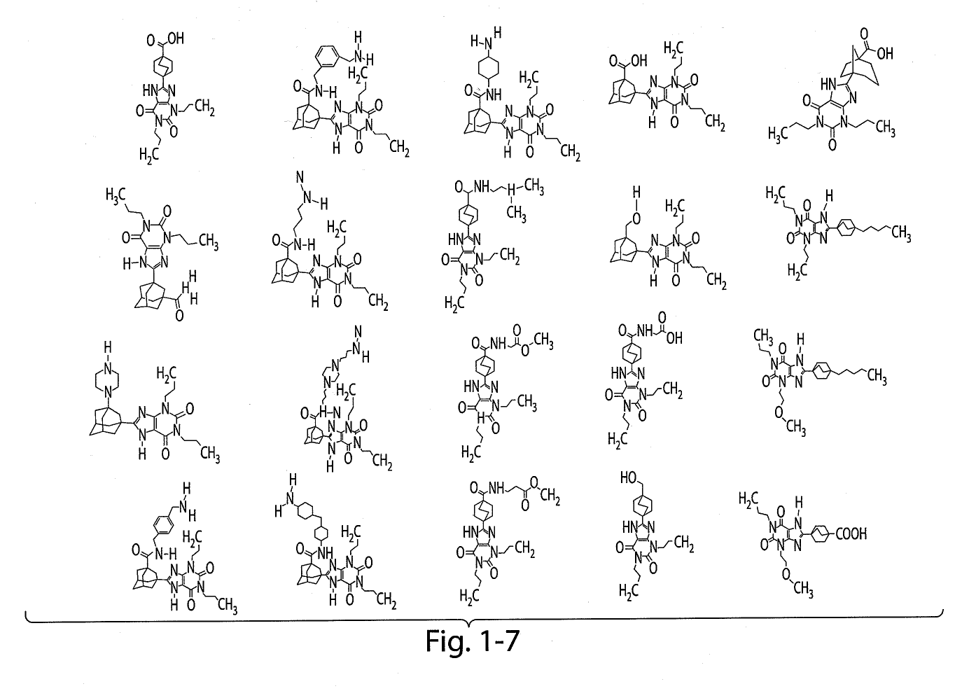

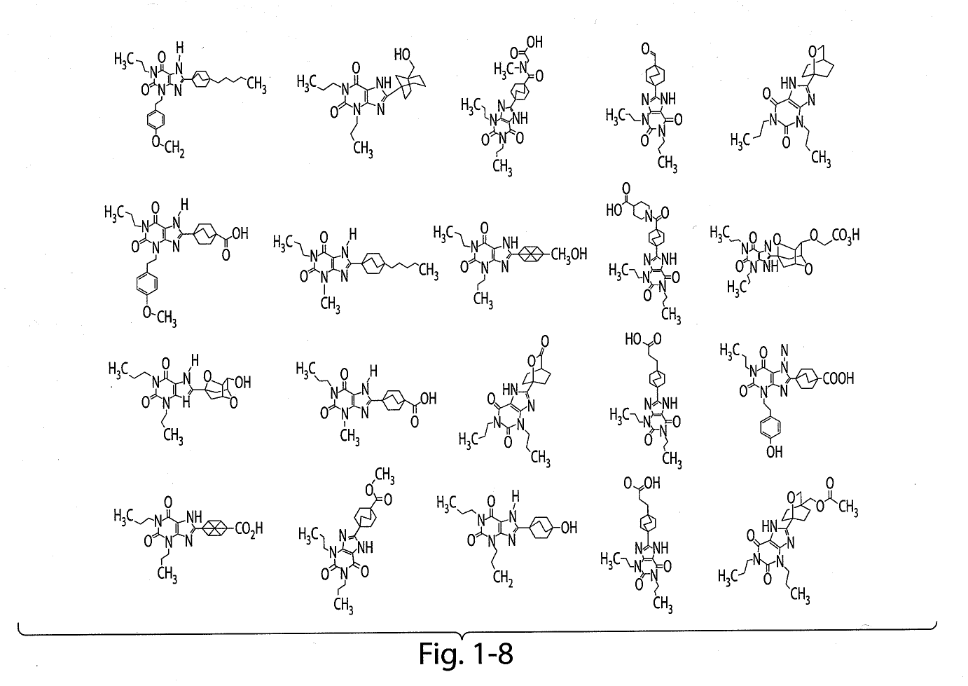

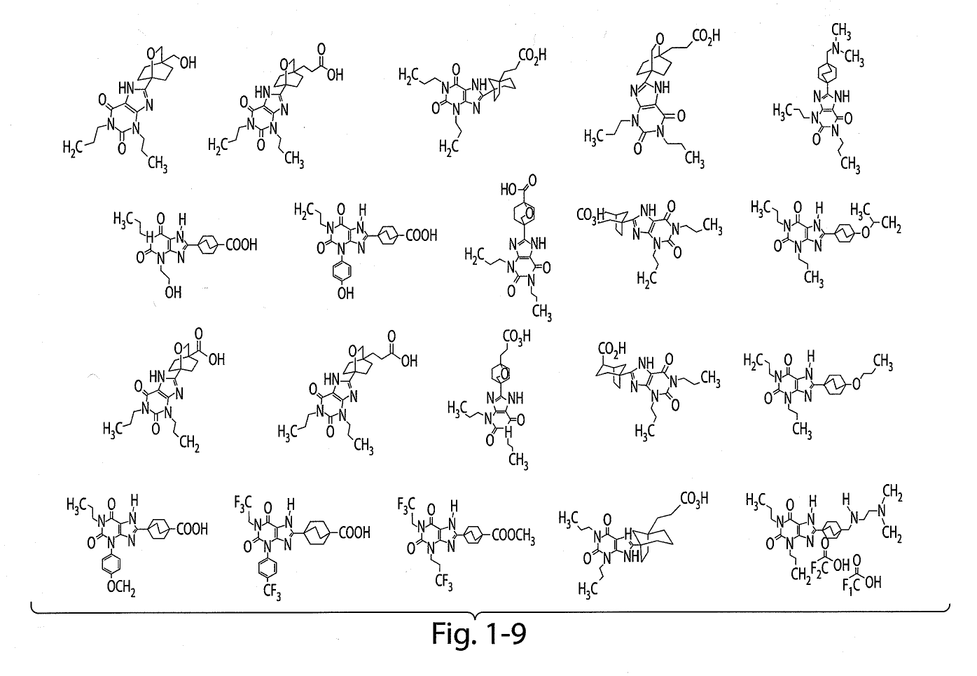

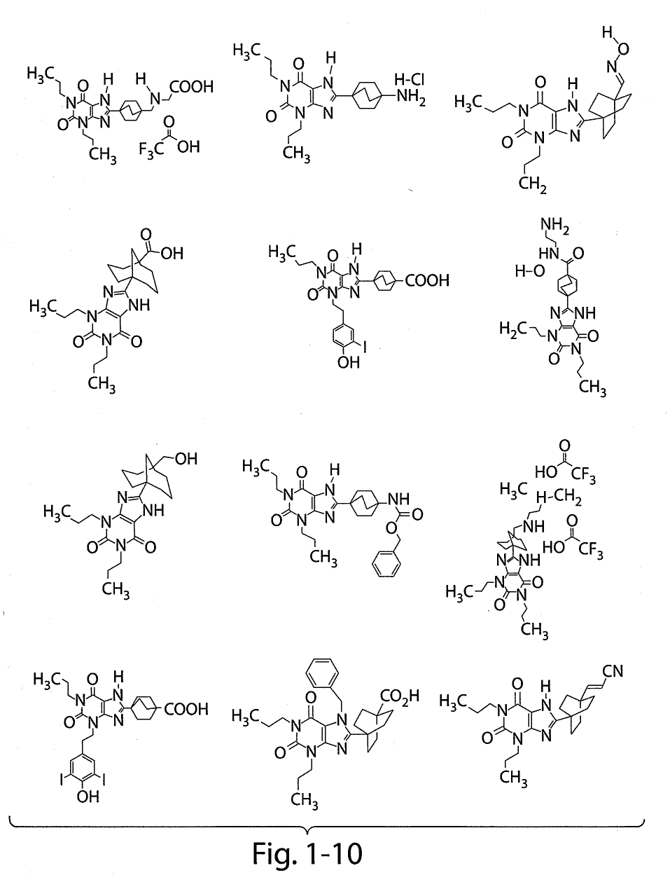

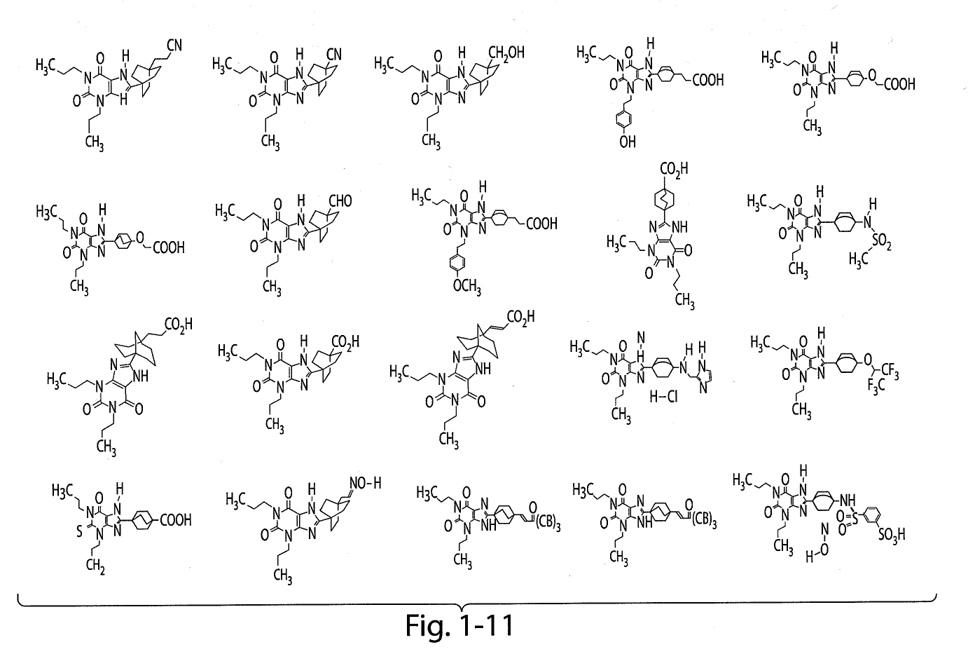

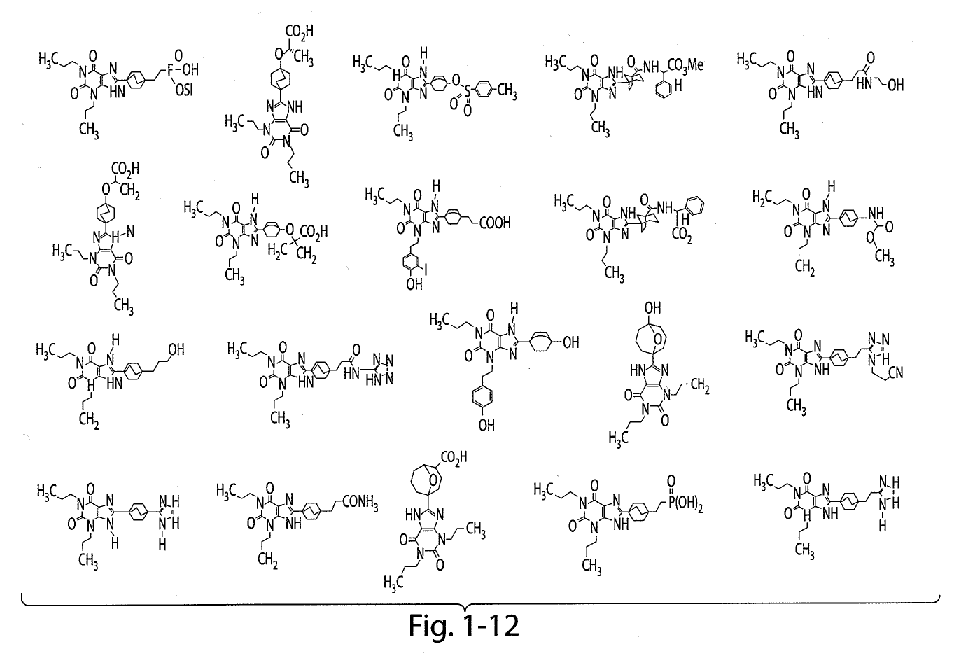

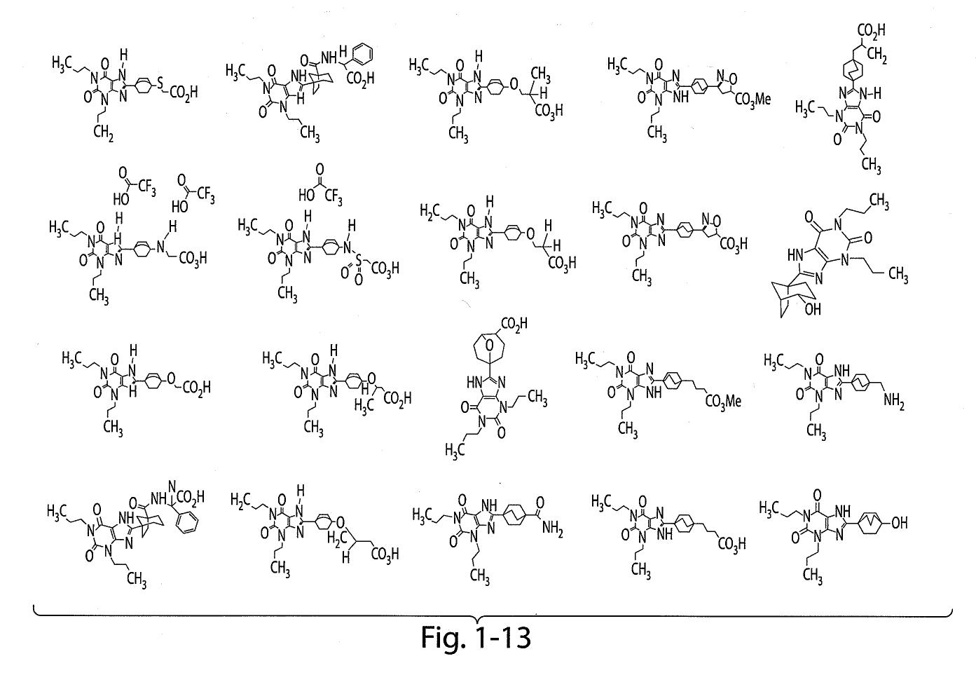

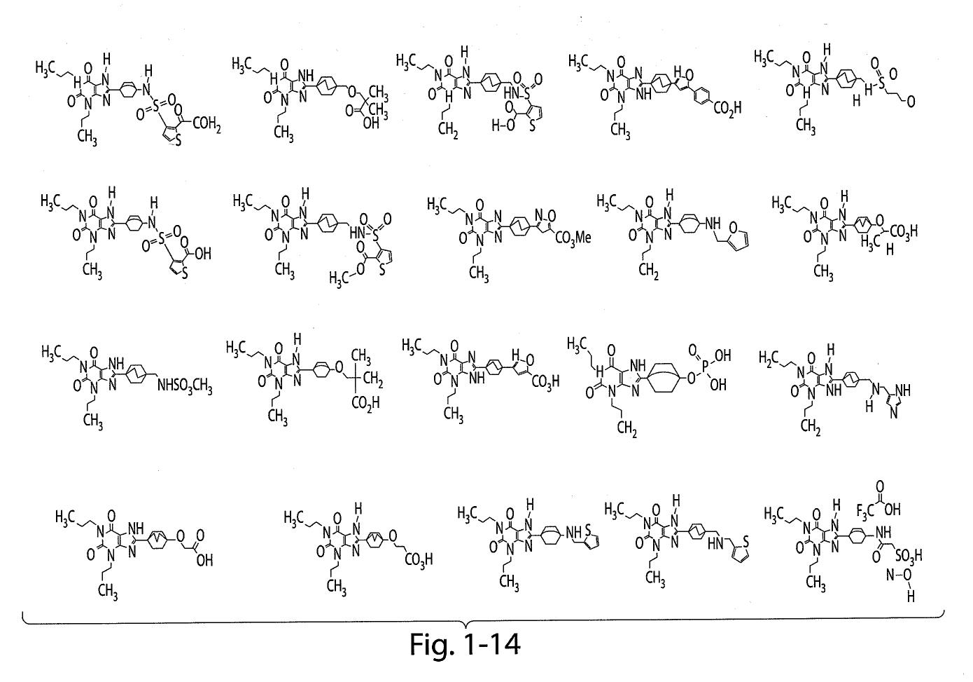

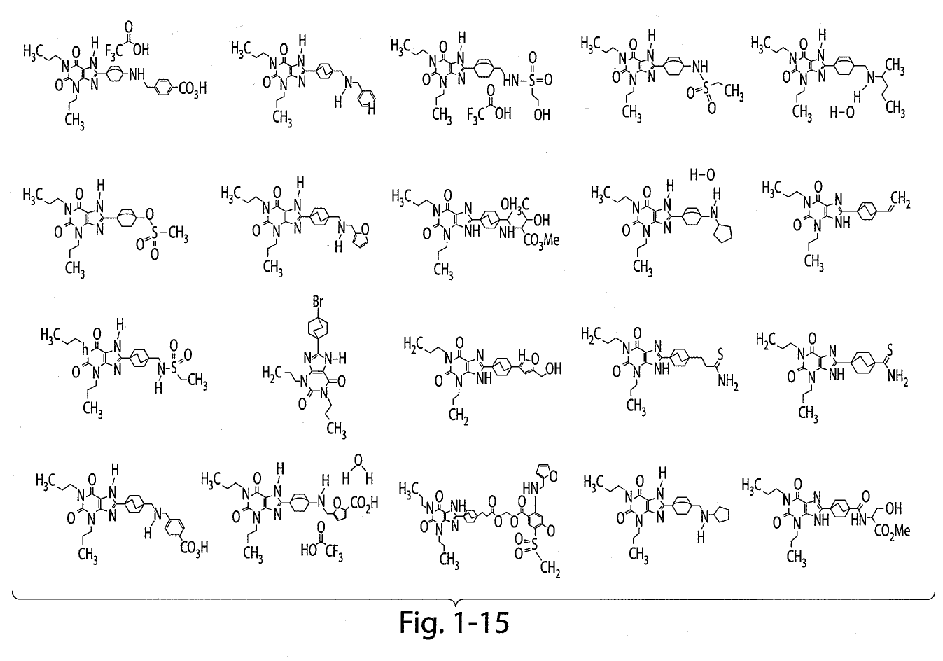

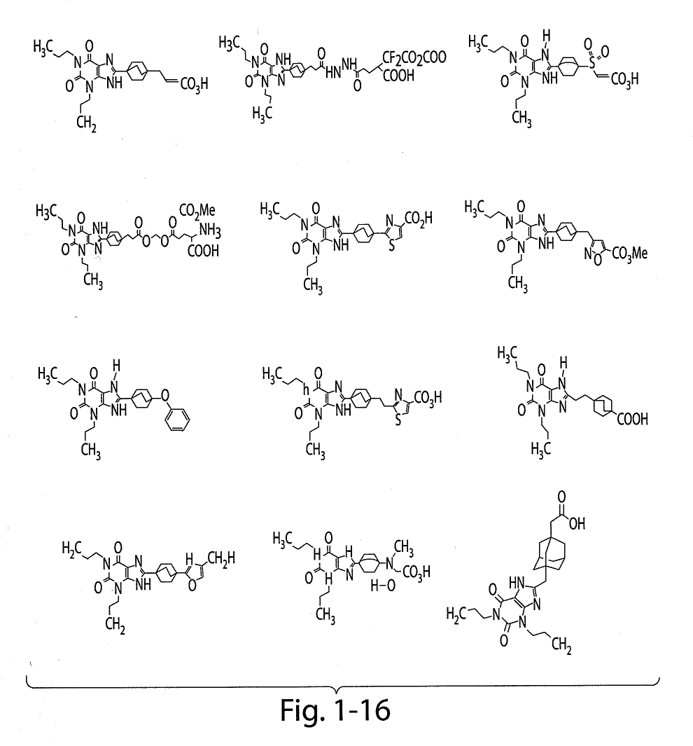

Additional exemplary adenosine receptor antagonists of formula I are provided in FIG. 1.

Exemplary adenosine receptor antagonists include compounds of formula II:

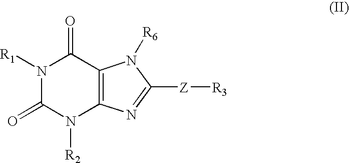

In formula I, R₁ and R₂ can, independently, be:

a) hydrogen;

b) alkyl, alkenyl of not less than 3 carbons, or alkynyl of not less than 3 carbons (e.g., where the alkyl, alkenyl, or alkynyl can be unsubstituted or can be functionalized with one or more substituents selected from hydroxy, alkoxy, amino, alkylamino, dialkylamino, heterocyclyl, acylamino, alkylsulfonylamino, and heterocyclylcarbonylamino; or

c) aryl or substituted aryl;

R₃ is selected from the group consisting of:

(a) a bicyclic, tricyclic or pentacyclic group selected from:

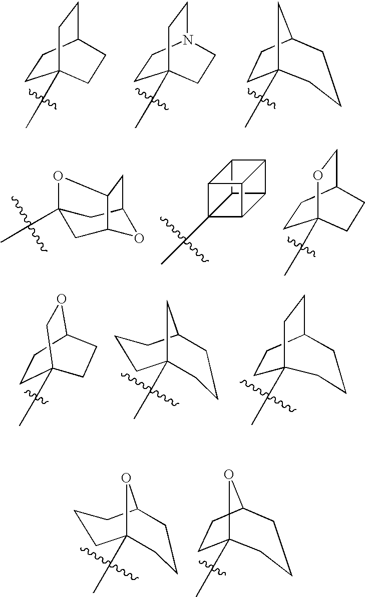

where the bicyclic or tricyclic group can be unsubstituted or can be functionalized with one or more substituents selected from:
(a) alkyl, alkenyl, and alkynyl; where each alkyl, alkenyl, or alkynyl group can be unsubstituted or can be functionalized with one or more substituents selected from the group consisting of (amino)(R₅)acylhydrazinylcarbonyl, (amino)(R₅)acyloxycarboxy, (hydroxy)(carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R₅, R₅-alkoxy, R₅-alkylamino, cyano, cyanoalkylcarbamoyl, cycloalkylamino, dialkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; or
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, cyano, cycloalkylamino, dialkylaminoalkylcarbamoyl, halogen, heterocyclyl, heterocyclylalkylamino, hydroxy, oximino, phosphate, substituted aralkylamino, substituted heterocyclyl, substituted heterocyclylsulfonylamino, sulfoxyacylamino, or thiocarbamoyl; and
(b) the tricyclic group:

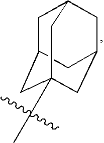

where the tricyclic group is functionalized with one or more substituents selected from the group consisting of:
(a) alkyl, alkenyl, and alkynyl; wherein each alkyl, alkenyl, or alkynyl group is either unsubstituted or functionalized with one or more substituents selected from the group consisting of (amino)(R₅)acylhydrazinylcarbonyl, (amino)(R₅)acyloxycarboxy, (hydroxy)(carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R₅, R₅-alkoxy, R₅-alkylamino, cyano, cyanoalkylcarbamoyl, cycloalkylamino, dialkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, cyano, cycloalkylamino, dialkylaminoalkylcarbamoyl, halogen, heterocyclyl, heterocyclylalkylamino, oximino, phosphate, substituted aralkylamino, substituted heterocyclyl, substituted heterocyclylsulfonylamino, sulfoxyacylamino, and thiocarbamoyl;

R₄ can be hydrogen, C₁₋₄-alkyl, C₁₋₄-alkyl-CO₂H, or phenyl; where the C₁₋₄-alkyl, C₁₋₄-alkyl-CO₂H, and phenyl groups can be unsubstituted or can be functionalized with one, two, three, or more substituents such as halogen, —OH, —OMe, —NH₂, —NO₂, and benzyl, or benzyl functionalized with one, two, three, or more substituents such as halogen, —OH, —OMe, —NH₂, and —NO₂;

R₅ can be —CH₂COOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, or any of the following:

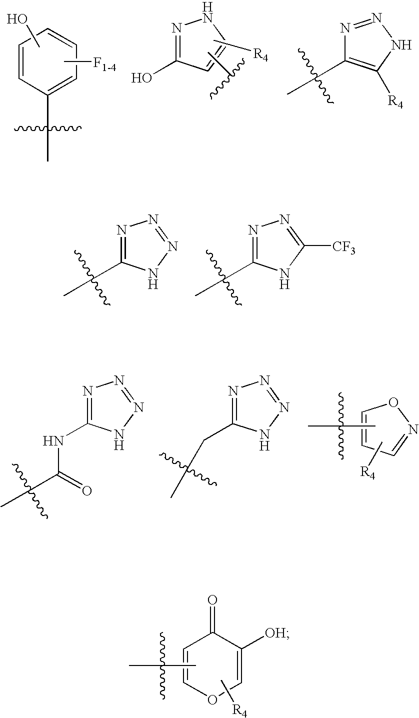

Z can be a single bond, —O—, —(CH₂)₁₋₃—, —O(CH₂)₁₋₂—, —CH₂OCH₂—, —(CH₂)₁₋₂O—, —CH═CHCH₂—, —CH═CH—, or —CH₂CH═CH—; and

R₆ can be hydrogen, alkyl, acyl, alkylsulfonyl, aralkyl, substituted aralkyl, substituted alkyl, or heterocyclyl.

In some embodiments, R₆ is hydrogen. However, when R₆ is methyl or another non-hydrogen substituent, the compounds can be highly selective for inhibition of adenosine Ata receptors.

In certain embodiments, R₁ and R₂ can be the same or different alkyl groups (e.g., one or both can be n-propyl).

R₃ can be aralkyl substituted with —OH, —OMe, or -halogen; -methyl; or 3-hydroxypropyl, and Z can be a single bond.

In some embodiments, R₃ can be:

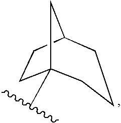

and can be unsubstituted or can be functionalized with one or more (i.e., 1, 2, 3, or more) substituents such as hydroxy, R₅—, or R₅-alkenyl. Thus, the compound can be, for example, 5-(2,6-Dioxo-1,3-dipropyl-2,3,6,7-tetrahydro-1H-purin-8-yl)-bicyclo[3.2.1]octane-1-carboxylic acid; 8-(4-Hydroxy-bicyclo[3.2.1]oct-1-yl)-1,3-dipropyl-3,7-dihydro-purine-2,6-dione; or 5-(2,6-Dioxo-1,3-dipropyl-2,3,6,7-tetrahydro-1H-purin-8-yl)-bicyclo[3.2.1]octane-2-carboxylic acid.

In other embodiments, R₃ can be:

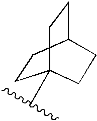

and can be unsubstituted or can be functionalized with one or more substituents such as hydroxy, R₅-alkyl, —R₅, R₅-alkenyl, alkoxycarbonyl, alkoxycarbonylalkyl, alkoxycarbonylalkenyl, hydroxyalkyl, aldehydo, alkoxyalkyl, R₅-alkoxy, phosphate, R₅-alkylcarbamoyl, and R₅-alkyl(alkyl)carbamoyl. Thus, the compound can be, for example, 8-(4-Hydroxy-bicyclo[2.2.2]oct-1-yl)-1,3-dipropyl-3,7-dihydro-purine-2,6-dione; 4-(2,6-Dioxo-1,3-dipropyl-2,3,6,7-tetrahydro-1H-purin-8-yl)-bicyclo[2.2.2]octane-1-carboxylic acid; 4-(2,6-Dioxo-1,3-dipropyl-2,3,6,7-tetrahydro-1H-purin-8-yl)-bicyclo[2.2.2]octane-1-carbaldehyde; 4-(2,6-Dioxo-1,3-dipropyl-2,3,6,7-tetrahydro-1H-purin-8-yl)-bicyclo[2.2.2]octane-1-carboxylic acid methyl ester; 3-[4-(2,6-Dioxo-1,3-dipropyl-2,3,6,7-tetrahydro-1H-purin-8-yl)-bicyclo[2.2.2]oct-1-yl]-acrylic acid methyl ester; 3-[4-(2,6-Dioxo-1,3-dipropyl-2,3,6,7-tetrahydro-1H-purin-8-yl)-bicyclo[2.2.2]oct-1-yl]-propionic acid methyl ester; 3-[4-(2,6-Dioxo-1,3-dipropyl-2,3,6,7-tetrahydro-1H-purin-8-yl)-bicyclo[2.2.2]oct-1-yl]-acrylic acid; 3-[4-(2,6-Dioxo-1,3-dipropyl-2,3,6,7-tetrahydro-1H-purin-8-yl)-bicyclo[2.2.2]oct-1-yl]-propionic acid; 4-[4-(2,6-Dioxo-1,3-dipropyl-2,3,6,7-tetrahydro-1H-purin-8-yl)-bicyclo[2.2.2]oct-1-yl]-butyric acid; Phosphoric acid mono-[4-(2,6-dioxo-1,3-dipropyl-2,3,6,7-tetrahydro-1H-purin-8-yl)-bicyclo[2.2.2]oct-1-yl]ester; {[4-(2,6-Dioxo-1,3-dipropyl-2,3,6,7-tetrahydro-1H-purin-8-yl)-bicyclo[2.2.2]octane-1-carbonyl]-methyl-amino}-acetic acid; {[4-(2,6-Dioxo-1,3-dipropyl-2,3,6,7-tetrahydro-1H-purin-8-yl)-bicyclo[2.2.2]octane-1-carbonyl]-amino}-acetic acid; 3-[4-(2,6-Dioxo-1,3-dipropyl-2,3,6,7-tetrahydro-1H-purin-8-yl)-bicyclo[2.2.2]oct-1-yloxy]-propionic acid; 3-[4-(2,6-Dioxo-1,3-dipropyl-2,3,6,7-tetrahydro-1H-purin-8-yl)-bicyclo[2.2.2]oct-1-yloxy]-propionic acid methyl ester; 3-[4-(2,6-Dioxo-1,3-dipropyl-2,3,6,7-tetrahydro-1H-purin-8-yl)-bicyclo[2.2.2]oct-1-yloxy]-propionic acid t-butyl ester; or 3-[4-(2,6-Dioxo-1,3-dipropyl-2,3,6,7-tetrahydro-1H-purin-8-yl)-bicyclo[2.2.2]oct-1-yl]-2-methyl-propionic acid. In some embodiments, the adenosine receptor antagonist is 3-[4-(2,6-Dioxo-1,3-dipropyl-2,3,6,7-tetrahydro-1H-purin-8-yl)-bicyclo[2.2.2]oct-1-yl]-propionic acid.

In another embodiment, R₃ can be:

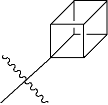

and can be unsubstituted or can be functionalized with one or more substituents such as R₅-alkyl, —R₅, R₅-alkenyl, alkoxycarbonyl, alkoxycarbonylalkenyl, hydroxyalkyl, aldehydo, and hydroxy. Thus, the compound can be, for example, 6-(2,6-Dioxo-1,3-dipropyl-2,3,6,7-tetrahydro-1H-purin-8-yl)-cubane-3-carboxylic acid; 8-(6-Hydroxymethyl-cuban-3-yl)-1,3-dipropyl-3,7-dihydro-purine-2,6-dione; or 3-[6-(2,6-Dioxo-1,3-dipropyl-2,3,6,7-tetrahydro-1H-purin-8-yl)-cuban-3-yl]-acrylic acid.

In yet another embodiment, R₃ can be:

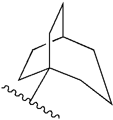

and can be unsubstituted or can be functionalized with one or more substituents such as R₅-alkyl, —R₅, R₅-alkenyl, R₅-alkoxy, alkoxycarbonyl, alkoxycarbonylalkenyl, hydroxyalkyl, aldehydo, and hydroxy. Thus, for example, the compound can be [5-(2,6-Dioxo-1,3-dipropyl-2,3,6,7-tetrahydro-1H-purin-8-yl)-bicyclo[3.2.2]non-1-yloxy]-acetic acid; 8-(5-Hydroxy-bicyclo[3.2.2]non-1-yl)-1,3-dipropyl-3,7-dihydro-purine-2,6-dione; or 5-(2,6-Dioxo-1,3-dipropyl-2,3,6,7-tetrahydro-1H-purin-8-yl)-bicyclo[3.2.2]nonane-1-carboxylic acid.

In still another embodiment, R₃ can be:

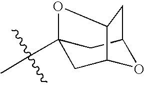

and can be unsubstituted or can be functionalized with one or more substituents such as hydroxy, R₅-alkoxy, R₅-alkenyl, alkoxycarbonyl, and carbonyl. Thus, for example, the compound can be 8-(4-Hydroxy-7-methyl-2,6-dioxa-bicyclo[3.3.1]non-1-yl)-1,3-dipropyl-3,7-dihydro-purine-2,6-dione; or [1-(2,6-Dioxo-1,3-dipropyl-2,3,6,7-tetrahydro-1H-purin-8-yl)-7-methyl-2,6-dioxa-bicyclo[3.3.1]non-4-yloxy]-acetic acid.

Additional exemplary adenosine receptor antagonists of formula II are provided in FIG. 1.

Additional exemplary adenosine receptor antagonists include compounds of formula III or IV:

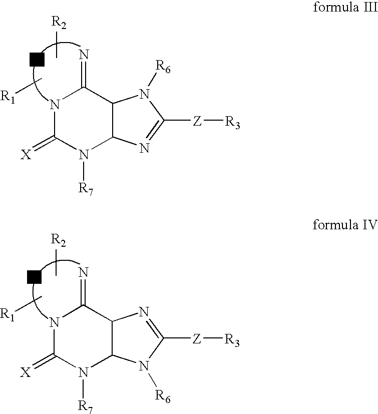

wherein R₁ and R₂ are independently selected from the group consisting of:

a) hydrogen;

b) alkyl, alkenyl or alkynyl, wherein said alkyl, alkenyl, or alkynyl is either unsubstituted or functionalized with one or more substituents selected from the group consisting of hydroxy, alkoxy, amino, alkylamino, dialkylamino, heterocyclyl, acylamino, alkylsulfonylamino, and heterocyclylcarbonylamino; and

c) aryl or substituted aryl;

R₃ is selected from the group consisting of:

(a) a bicyclic, tricyclic or pentacyclic group selected from the group consisting of:

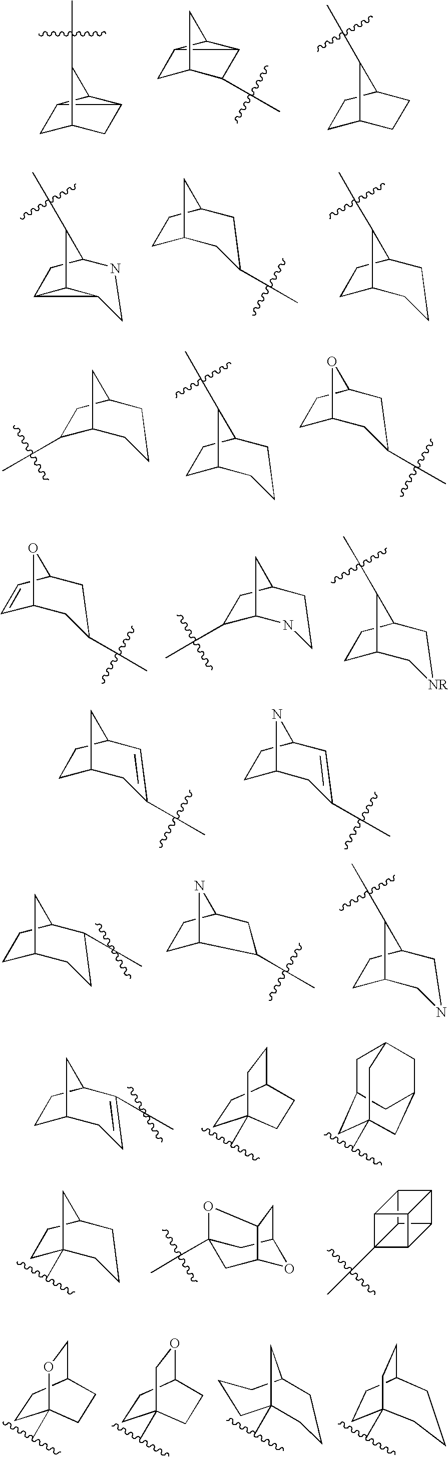

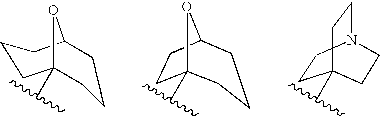

wherein the bicyclic, tricyclic or pentacyclic group is either unsubstituted or functionalized with one or more substituents selected from the group consisting of:
(i) alkyl, alkenyl and alkynyl; wherein each alkyl, alkenyl or alkynyl group is either unsubstituted or functionalized with one or more substituents selected from the group consisting of (alkoxycarbonyl)aralkylcarbamoyl, (amino)(R₅)acylhydrazinylcarbonyl, (amino)(R₅)acyloxycarboxy, (hydroxy)(carboalkoxy)alkylcarbamoyl, acylaminoalkylamino, acyloxy, aldehydo, alkenoxy, alkenylamino, alkenylsulfonylamino, alkoxy, alkoxycarbonyl, alkoxycarbonylalkylamino, alkoxycarbonylamino, alkoxycarbonylaminoacyloxy, alkoxycarbonylaminoalkylamino, alkylamino, alkylaminoalkylamino, alkylcarbamoyl, alkylphosphono, alkylsulfonylamino, alkylsulfonyloxy, amino, aminoacyloxy, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonyl, aralkoxycarbonylamino, arylheterocyclyl, aryloxy, arylsulfonylamino, arylsulfonyloxy, carbamoyl, carbonyl, cyano, cyanoalkylcarbamoyl, cycloalkylamino, dialkylamino, dialkylaminoalkylamino, dialkylaminoalkylcarbamoyl, dialkylphosphono, haloalkylsulfonylamino, halogen, heterocyclyl, heterocyclylalkylamino, heterocyclylcarbamoyl, hydroxy, hydroxyalkylsulfonylamino, oximino, phosphate, phosphono, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, substituted aralkylamino, substituted arylcarboxyalkoxycarbonyl, substituted arylsulfonylaminoalkylamino, substituted heteroarylsulfonylamino, substituted heterocyclyl, substituted heterocyclylaminoalkylamino, substituted heterocyclylsulfonylamino, sulfoxyacylamino, thiocarbamoyl, trifluoromethyl; and
(ii) (alkoxycarbonyl)aralkylcarbamoyl, (amino)(R₅)acylhydrazinylcarbonyl, (amino)(R₅)acyloxycarboxy, (hydroxy)(carboalkoxy)alkylcarbamoyl, acylaminoalkylamino, acyloxy, aldehydo, alkenoxy, alkenylamino, alkenylsulfonylamino, alkoxy, alkoxycarbonyl, alkoxycarbonylalkylamino, alkoxycarbonylamino, alkoxycarbonylaminoacyloxy, alkoxycarbonylaminoalkylamino, alkylamino, alkylaminoalkylamino, alkylcarbamoyl, alkylphosphono, alkylsulfonylamino, alkylsulfonyloxy, amino, aminoacyloxy, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonyl, aralkoxycarbonylamino, arylheterocyclyl, aryloxy, arylsulfonylamino, arylsulfonyloxy, carbamoyl, carbonyl, cyano, cyanoalkylcarbamoyl, cycloalkylamino, dialkylamino, dialkylaminoalkylamino, dialkylaminoalkylcarbamoyl, dialkylphosphono, haloalkylsulfonylamino, halogen, heterocyclyl, heterocyclylalkylamino, heterocyclylcarbamoyl, hydroxy, hydroxyalkylsulfonylamino, oximino, phosphate, phosphono, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, substituted aralkylamino, substituted arylcarboxyalkoxycarbonyl, substituted arylsulfonylaminoalkylamino, substituted heteroarylsulfonylamino, substituted heterocyclyl, substituted heterocyclylaminoalkylamino, substituted heterocyclylsulfonylamino, sulfoxyacylamino, thiocarbamoyl, trifluoromethyl;

R₄ is selected from the group consisting of hydrogen, C₁₋₄-alkyl, C₁₋₄-alkyl-CO₂H, and phenyl, wherein the C₁₋₄-alkyl, C₁₋₄-alkyl-CO₂H, and phenyl groups are either unsubstituted or functionalized with one to three substituents selected from the group consisting of halogen, —OH, —OMe, —NH₂, NO₂, benzyl, and benzyl functionalized with one to three substituents selected from the group consisting of halogen, —OH, —OMe, —NH₂, and —NO₂;

R₅ is selected from the group consisting of —(CR₁R₂)ₙCOOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, and the following:

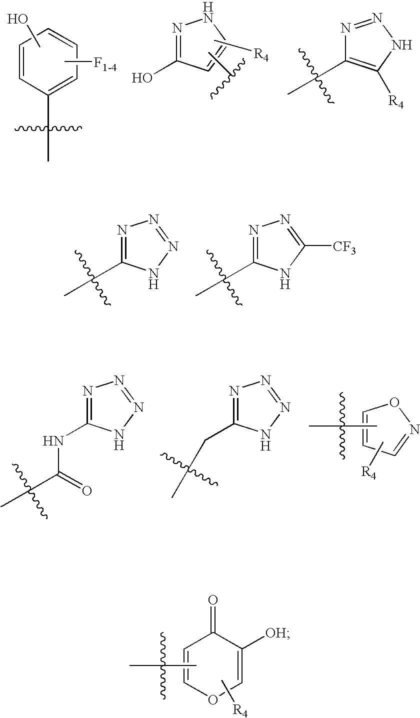

n=0, 1, 2 or 3;

A is selected from the group consisting of —CH═CH, —(CH)ₘ—(CH)ₘ, CH═CH—CH₂, and —CH₂—CH═CH;

m=1 or 2;

X is O or S;

Z is selected from the group consisting of a single bond, —O—, —(CH₂)ₙ—, —O(CH₂)₁₋₂—, —CH₂OCH₂—, —(CH₂)₁₋₂O—, —CH═CHCH₂—, —CH═CH—, and —CH₂CH═CH—; and

R₆ is selected from the group consisting of hydrogen, alkyl, acyl, alkylsulfonyl, aralkyl, substituted aralkyl, substituted alkyl, and heterocyclyl; and

R₇ is selected from the group consisting of:

a) hydrogen;

b) alkyl, alkenyl of not less than 3 carbons, or alkynyl of not less than 3 carbons; wherein said alkyl, alkenyl or alkynyl is either unsubstituted or functionalized with one or more substitutents selected from the group consisting of hydroxy, alkoxy, amino, alkylamino, dialkylamino, heterocyclyl, acylamino, alkylsulfonylamino, and heterocyclylcarbonylamino; and

c) aryl or substituted aryl;

d) alkylaryl or alkyl substituted aryl.

The compounds of Formula I or II optionally can be in forms such as an achiral compound, a racemate, an optically active compound, a pure diastereomer, a mixture of diastereomers, or a pharmacologically acceptable addition salt. In certain embodiments, the compounds of the invention are compounds of Formula I or II wherein neither of R₁ and R₂ are hydrogen, that is, each of R₁ and R₂ are independently selected from the group consisting of

a) alkyl, alkenyl or alkynyl, wherein said alkyl, alkenyl, or alkynyl is either unsubstituted or functionalized with one or more substituents selected from the group consisting of hydroxy, alkoxy, amino, alkylamino, dialkylamino, heterocyclyl, acylamino, alkylsulfonylamino, and heterocyclylcarbonylamino; and

b) aryl or substituted aryl.

In some embodiments, at least one of R₁ and R₂ is alkyl. In yet other preferred embodiments,

A is —(CH)ₘ—(CH)ₘ.

In some embodiments, R₇ is alkyl, and Z is a single bond.

Exemplary compounds of this invention are:
2-(4-Hydroxy-bicyclo[2.2.2]oct-1-yl)-7-isopropyl-4-propyl-1,4,6,7-tetrahydro-1,3,4,5a,8-pentaaza-as-indacen-5-one
7-Ethyl-2-(4-hydroxy-bicyclo[2.2.2]oct-1-yl)-4-propyl-1,4,6,7-tetrahydro-1,3,4,5a,8-pentaaza-as-indacen-5-one;
3-[4-(7-Ethyl-5-oxo-4-propyl-4,5,6,7-tetrahydro-1H-1,3,4,5a,8-pentaaza-as-indacen-2-yl)-bicyclo[2.2.2]oct-1-yl]-propionic acid;
2-(4-Hydroxy-bicyclo[2.2.2]oct-1-yl)-7-methyl-4-propyl-1,4,6,7-tetrahydro-1,3,4,5a,8-pentaaza-as-indacen-5-one; and
3-[4-(7-Isopropyl-5-oxo-4-propyl-4,5,6,7-tetrahydro-1H-1,3,4,5a,8-pentaaza-as-indacen-2-yl)-bicyclo[2.2.2]oct-1-yl]-propionic acid.

Exemplary compounds are provided in Table 1 below:

TABLE 1

| Compound | Structure |
|---|---|
| 1 | 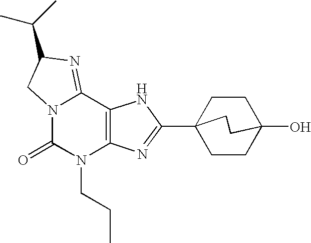 |
| 2 | 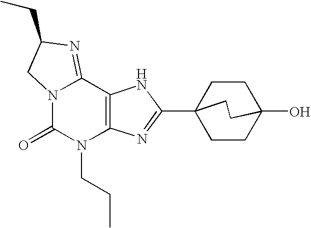 |
| 3 | 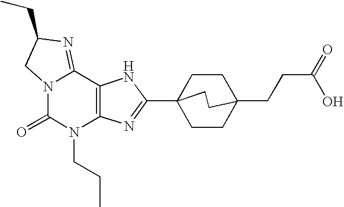 |
| 4 | 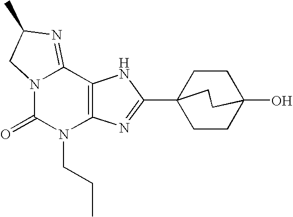 |
| 5 | 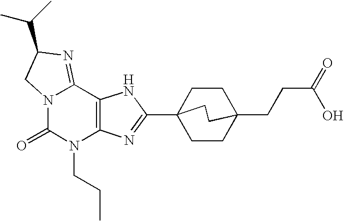 |
| 6 | 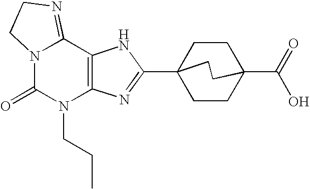 |
| 7 | 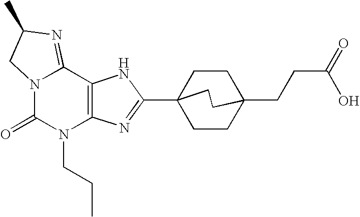 |
| 8 | 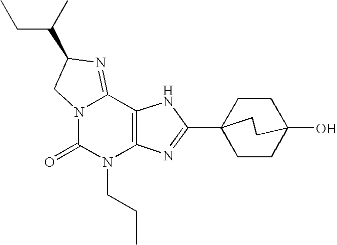 |
| 9 | 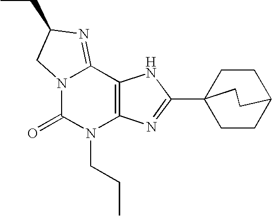 |
| 10 | 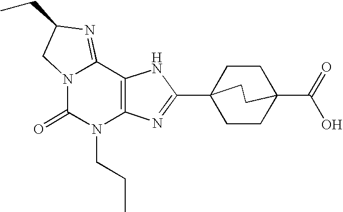 |
| 11 | 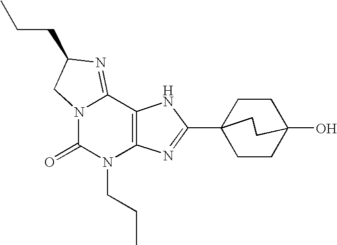 |
| 12 | 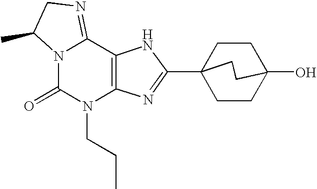 |
| 13 | 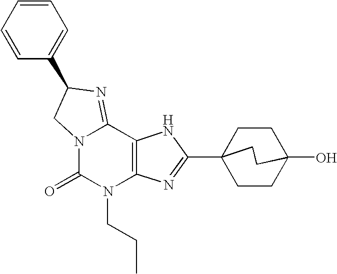 |
| 14 | 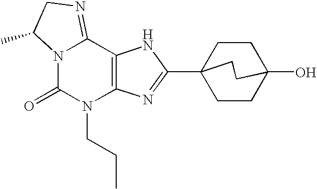 |
| 15 | 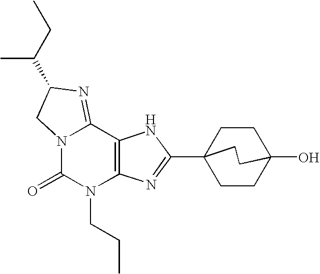 |
| 16 | 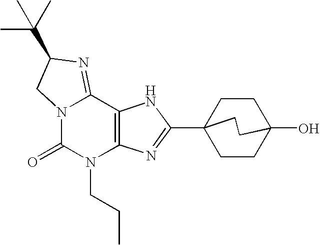 |
| 17 | 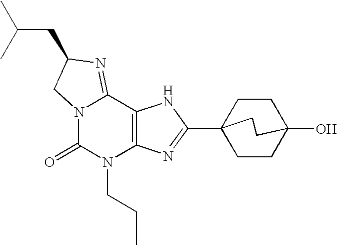 |
| 18 | 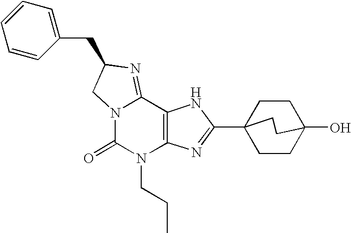 |
| 19 | 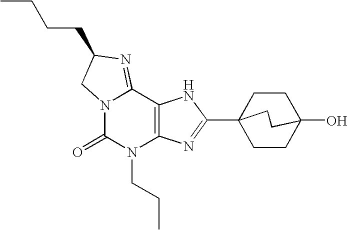 |
| 20 | 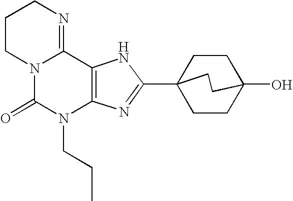 |
| 21 | 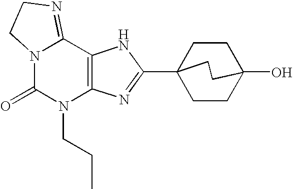 |
| 22 | 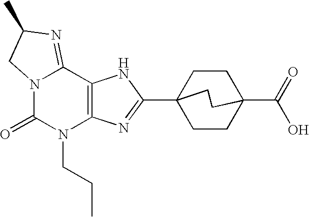 |
| 23 | 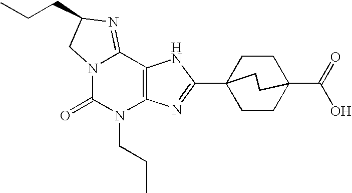 |
| 24 | 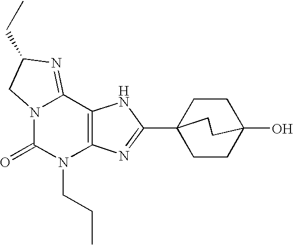 |
| 25 | 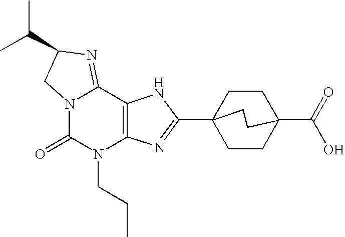 |
| 26 | 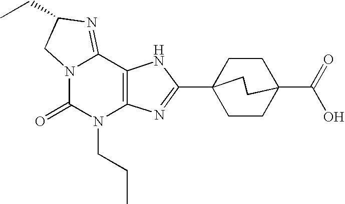 |
| 27 | 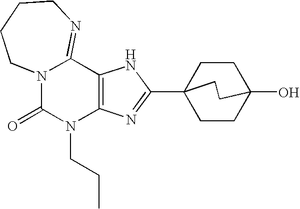 |
| 28 | 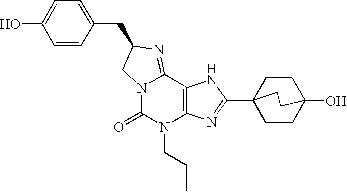 |
| 29 | 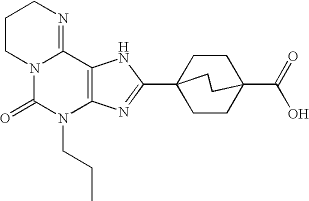 |
| 30 | 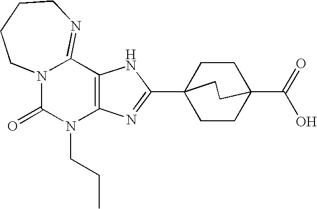 |
| 31 | 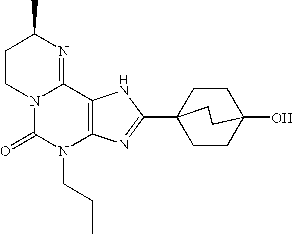 |
| 32 | 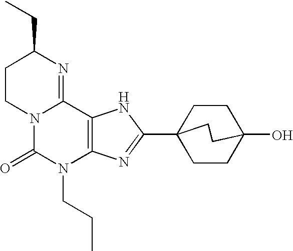 |

The compound can be, for example, in a form of an achiral compound, a racemate, an optically active compound, a pure diastereomer, a mixture of diastereomers, or a pharmacologically acceptable addition salt.

The compounds described herein can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

The compounds described herein can be made using synthetic methods known in the art and described, for example, in U.S. Pat. No. 6,605,600, U.S. Pat. No. 6,649,600, and U.S. Pat. No. 6,605,601, each of which are incorporated by reference herein.

As used herein, an “alkyl” group is a saturated aliphatic hydrocarbon group. An alkyl group can be straight or branched, and can have, for example, from 1 to 6 carbon atoms in a chain. Examples of straight chain alkyl groups include, but are not limited to, ethyl and butyl. Examples of branched alkyl groups include, but are not limited to, isopropyl and t-butyl.

An “alkenyl” group is an aliphatic carbon group that has at least one double bond. An alkenyl group can be straight or branched, and can have, for example, from 3 to 6 carbon atoms in a chain and 1 or 2 double bonds. Examples of alkenyl groups include, but are not limited to, allyl and isoprenyl.

An “alkynyl” group is an aliphatic carbon group that has at least one triple bond. An alkynyl group can be straight or branched, and can have, for example, from 3 to 6 carbon atoms in a chain and 1 to 2 triple bonds. Examples of alkynyl groups include, but are not limited to, propargyl and butynyl.

An “aryl” group is a phenyl or naphthyl group, or a derivative thereof. A “substituted aryl” group is an aryl group that is substituted with one or more substituents such as alkyl, alkoxy, amino, nitro, carboxy, carboalkoxy, cyano, alkylamino, dialkylamino, halo, hydroxy, hydroxyalkyl, mercaptyl, alkylmercaptyl, trihaloalkyl, carboxyalkyl, sulfoxy, or carbamoyl.

An “aralkyl” group is an alkyl group that is substituted with an aryl group. An example of an aralkyl group is benzyl.

A “cycloalkyl” group is an aliphatic ring of, for example, 3 to 8 carbon atoms. Examples of cycloalkyl groups include cyclopropyl and cyclohexyl.

An “acyl” group is a straight or branched alkyl-C(═O)— group or a formyl group. Examples of acyl groups include alkanoyl groups (e.g., having from 1 to 6 carbon atoms in the alkyl group). Acetyl and pivaloyl are examples of acyl groups. Acyl groups may be substituted or unsubstituted.

A “carbamoyl” group is a group having the structure H₂N—CO₂—. “Alkylcarbamoyl” and “dialkylcarbamoyl” refer to carbamoyl groups in which the nitrogen has one or two alkyl groups attached in place of the hydrogens, respectively. By analogy, “arylcarbamoyl” and “arylalkylcarbamoyl” groups include an aryl group in place of one of the hydrogens and, in the latter case, an alkyl group in place of the second hydrogen.

A “carboxyl” group is a —COOH group.

An “alkoxy” group is an alkyl-O— group in which “alkyl” is as previously described.

An “alkoxyalkyl” group is an alkyl group as previously described, with a hydrogen replaced by an alkoxy group, as previously described.

A “halogen” or “halo” group is fluorine, chlorine, bromine or iodine.

A “heterocyclyl” group is a 5 to about 10 membered ring structure, in which one or more of the atoms in the ring is an element other than carbon, e.g., N, O, S. A heterocyclyl group can be aromatic or non-aromatic, i.e., can be saturated, or can be partially or fully unsaturated. Examples of heterocyclyl groups include pyridyl, imidazolyl, furanyl, thienyl, thiazolyl, tetrahydrofuranyl, tetrahydropyranyl, morpholinyl, thiomorpholinyl, indolyl, indolinyl, isoindolinyl, piperidinyl, pyrimidinyl, piperazinyl, isoxazolyl, isoxazolidinyl, tetrazolyl, and benzimidazolyl.

A “substituted heterocyclyl” group is a heterocyclyl group wherein one or more hydrogens are replaced by substituents such as alkoxy, alkylamino, dialkylamino, carbalkoxy, carbamoyl, cyano, halo, trihalomethyl, hydroxy, carbonyl, thiocarbonyl, hydroxyalkyl or nitro.

A “hydroxyalkyl” means an alkyl group substituted by a hydroxy group.

A “sulfamoyl” group has the structure —S(O)₂NH₂. “Alkylsulfamoyl” and “dialkylsulfamoyl” refer to sulfamoyl groups in which the nitrogen has one or two alkyl groups attached in place of the hydrogens, respectively. By analogy, “arylsulfamoyl” and “arylalkylsulfamoyl” groups include an aryl group in place of one of the hydrogens and, in the latter case, an alkyl group in place of the second hydrogen.

Pharmaceutical Compositions and Dosage Forms

The adenosine receptor antagonists described herein, e.g., 3-[4-(2,6-Dioxo-1,3-dipropyl-2,3,6,7-tetrahydro-1H-purin-8-yl)-bicyclo[2.2.2]oct-1-yl]-propionic acid, are generally prepared in a composition, such as a pharmaceutically acceptable composition. In some embodiments, the pharmaceutical composition is a composition that can be administered to a subject parenterally (e.g., a liquid composition such as a solution). In some embodiments, the composition is a solid composition, for example, a lyophilite, which can be further processed prior to administering the composition to a subject, for example, the solid composition can be further processed to form a liquid composition such as a solution.

Liquid Compositions

Liquid compositions including an adenosine receptor antagonist described herein such as 3-[4-(2,6-Dioxo-1,3-dipropyl-2,3,6,7-tetrahydro-1H-purin-8-yl)-bicyclo[2.2.2]oct-1-yl]-propionic acid are generally prepared for parental administration to the subject. In addition to the adenosine receptor antagonists described herein, e.g., 3-[4-(2,6-Dioxo-1,3-dipropyl-2,3,6,7-tetrahydro-1H-purin-8-yl)-bicyclo[2.2.2]oct-1-yl]-propionic acid, in some embodiments, the liquid compositions also include additional components such as a buffer, a carrier, and/or a vehicle.

The pharmaceutical compositions described herein can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation. In some embodiments, a pharmaceutical composition described herein includes mannitol and/or water.

A composition can be in the form of an aqueous solution. In some embodiments, the composition is maintained at a pH of at least about 8, e.g., about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, or greater. In some embodiments, the pH of the composition is maintained at least about 8.0 e.g., from about 8.0 to about 10.0 or from about 8.5 to about 9.5 e.g., about 8.5 or about 9.5. The desired pH can be maintained, for example, by including a buffer in the composition. In some embodiments, the composition includes a histidine buffer. The molar ratio of adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid to histidine is generally from about 5:1 to about 0.5:1 e.g., from about 3:1 to about 1:1, for example, about 2.5:1, about 2:1, or about 1.5:1. In some embodiments, the composition also includes a base, for example, sodium hydroxide.

The concentration of an adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid is generally from about 0.05 mM to about 10 mM e.g., about 0.1 mM to about 10 mM, for example, about 1 mM. In some embodiments, the concentration of the adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid is from about 0.05 mM to about 5 mM, for example, about 0.5 mM.

Solid Compositions

Solid compositions, for example, compositions that can be further processed into a composition for parental administration are also included herein. In some embodiments, a composition is in the form of a solid powder, such as a lyophilite, which can be further processed, for example, reconstituted, to form a solution for parental administration to a subject. The solid compositions can include a buffer and/or a carrier, for example, as described for the liquid compositions above.

The solid compositions described herein can be mixed with a liquid vehicle, e.g., water, to provide a composition for parental administration. In some embodiments, the composition for parental administration is a sterile injectable solution.

In some embodiments, the composition is provided such that, when reconstituted with a vehicle, the resulting composition has a pH of at least about 9, e.g., about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, or greater, e.g., about 9.5. The desired pH can be maintained, for example, by including a buffer in the composition. In some embodiments, the composition includes a histidine buffer. The molar ratio of adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid to histidine is generally from about 5:1 to about 0.5:1 e.g., from about 3:1 to about 1:1, for example, about 2.5:1, about 2:1, or about 1.5:1. In some embodiments, the composition also includes a base, for example, sodium hydroxide.

When a solid composition described herein is combined with a vehicle to provide a composition for parental administration, the amount of vehicle added to the composition is generally in an amount so as to provide a concentration of an adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid of from about 0.05 mM to about 10 mM e.g., about 0.1 mM to about 10 mM, for example, about 1 mM. In some embodiments, the concentration of the adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid is from about 0.05 mM to about 5 mM, for example, about 0.5 mM.

Dosage Forms

The compositions described herein can be provided in single dosage forms. Exemplary dosage forms provide from about 1 to about 75 mg of adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid, e.g., 2 mg to about 60 mg. For example, the single dosage form can include about 2 mg, about 10 mg, about 20 mg, or about 60 mg of the adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid.

In some embodiments, the compositions described herein include a buffer such as histidine. When present, molar ratio of adenosine receptor antagonist such as a compound described herein, e.g., 3-[4-(2,6-Dioxo-1,3-dipropyl-2,3,6,7-tetrahydro-1H-purin-8-yl)-bicyclo[2.2.2]oct-1-yl]-propionic acid to histidine if generally from about 5:1 to about 0.5:1 e.g., from about 3:1 to about 1:1, for example, about 2.5:1, about 2:1, or about 1.5:1.

Exemplary single dosage forms generally include from about 0.2 mg to about 15 mg of histidine e.g., about 0.4 mg to about 12 mg, for example, about 0.4 mg, about 2 mg, about 4 mg, or about 12 mg. In some embodiments, the dosage form also includes sodium hydroxide. In some embodiments, a single dosage form includes about 25 ml of a vehicle such as water, e.g., about 50 ml, about 75 ml, or about 100 ml. Exemplary singe dosage forms include dosages of from about 0.03 mg/kg to about 3.0 mg/kg, e.g., about 0.03 mg/kg, 0.15 mg/kg, 0.3 mg/kg, 1 mg/kg, or 3 mg/kg.

As with the pharmaceutical compositions above, the single dosage forms can also include additional components such as a buffer, carrier, and/or vehicle. In some embodiments, the dosage form includes mannitol and/or water. In some embodiments, a single dosage form includes about 25 ml of a vehicle such as water, e.g., about 50 ml, about 75 ml, or about 100 ml.

Kits

A compound described herein can be provided in a kit, for example, wherein the compound is provided in any form described herein, e.g., liquid or solid e.g., lyophilized form. In some embodiments, a compound described herein is substantially pure and/or sterile. When a compound described herein is provided in a liquid solution, the liquid solution can be an aqueous solution, e.g., a sterile aqueous solution. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

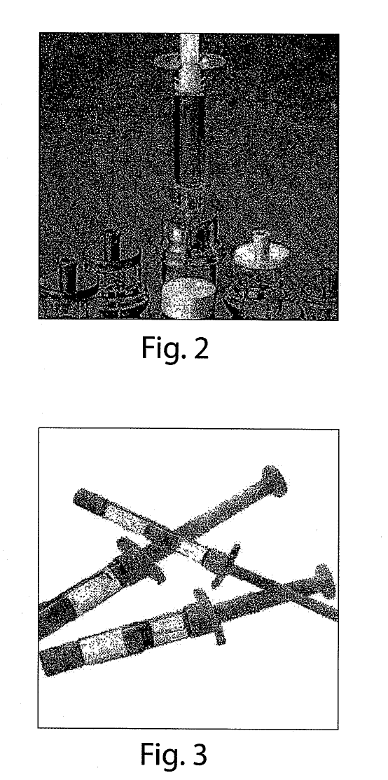

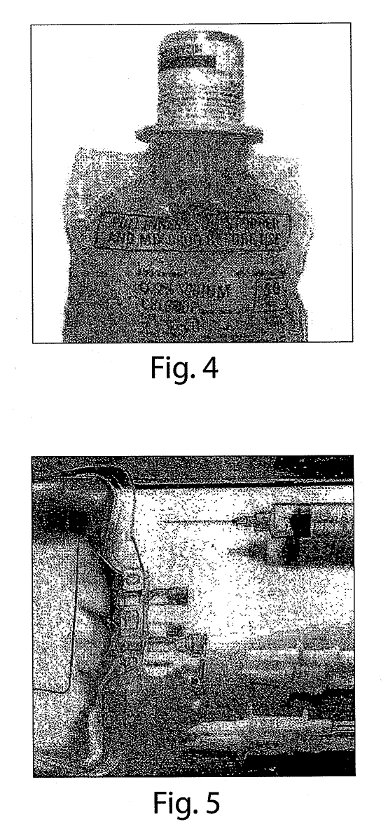

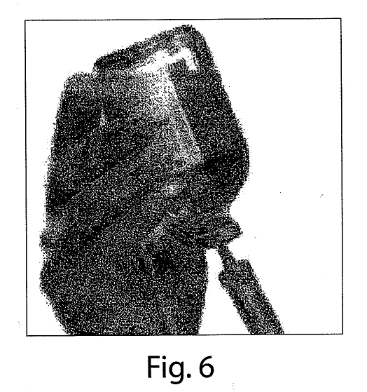

The kit optionally includes a device suitable for administration of the composition, e.g., a syringe or any such delivery device. Exemplary kits are provided in FIGS. 2-6.

Methods of Administration

Selection of a Patient

Methods are described herein wherein an adenosine receptor antagonist is administered to a subject in need thereof. For example, in some embodiments, an adenosine receptor antagonist is administered to a subject wherein an ischemic event is imminent. Exemplary ischemic events include acute coronary syndrome, stroke, organ transplantation (e.g., organ transplantation surgery), kidney ischemia, or shock. In some embodiments, an adenosine receptor antagonist is administered within two days before or after an ischemic event (e.g., within two days after an ischemic event).

In some embodiments, administration of an adenosine receptor antagonist, for example, a compound or composition described herein, can increase the risk in a subject of a seizure. In some embodiments, the increased risk of seizure can be reduced, for example, by modification of the timing (e.g., twice daily administration) and or method of administration (e.g., administration by infusion) of the adenosine receptor antagonist.

In some embodiments, a patient is selected wherein the patient does not have an elevated risk of seizure or is at low risk of a seizure prior to administration of the adenosine receptor antagonist (e.g., the patient is selected, at least in part, on this basis). For example, the subject does not have a history of past seizures (no struck or head trauma within the past six months, no seizure within the last ten years, and/or the subject has not been on any anti-seizure medication for the past five years. In some embodiments, upon selection of the patient, the patient is administered a compound or composition described herein. The patient can be evaluated for one or more parameters prior to administration of the adenosine receptor antagonist.

In some embodiments, a patient is selected wherein the patient does not meet one or more of the following criteria (e.g., the patient is selected, at least in part, on the basis of not having one or more of the following criteria): the patient requires more than short-acting nitrates to treat angina or the patient has had unstable angina; the patient has renal impairment (e.g., 3 g proteinuria in 24 hours or CrCl, 30 m./min/1.73 m²), a potential for recent renal injury or a history of urinary obstruction; or the patient has a history of myocardial infarction or stroke within the prior three months, coronary artery bypass graft or percutaneous coronary intervention performed or planned within eight weeks of baseline, or another serious systemic disease.

In some embodiments a patient is selected such that, prior to administration, the patient has a glomerular filtration rate (GFR) of from about 10 to about 80, e.g., from about 20 to about 70, the patient is identified as having class II-IV heart failure, the patient has a body mass index of greater than or equal to 19 kg/m2 and a body weight of less than or equal to 133 kg, and/or is being treated with an additional agent such as an ACE inhibitor and/or an angiotensin II receptor blocker (e.g., on a stable dose for at least seven days prior to administration of the adenosine receptor antagonist). The patient can be evaluated for one or more parameters prior to administration of the adenosine receptor antagonist.

Class II-IV heart failure refers to classifications of heart failure by the New York Heart Association. Class II is characterized as mild chronic heart failure, generally associated with slight limitation of physical activity (e.g., ordinary physical activity generally results in fatigue, palpitation, dyspnea or angina pectoris). Class III is characterized as moderate chronic heart failure, generally associated with a marked limitation of physical activity (e.g., less than ordinary physical activity generally leads to symptoms). Class IV is characterized as severe chronic heart failure, generally associated with being unable to carry on any physical activity without discomfort (e.g., symptoms of chronic heart failure generally present at rest). (See e.g., Nomenclature and Criteria for Diagnosis of Diseases of the Heart and Great Vessels (Little, Brown & Co). The ninth edition, revised by the Criteria Committee of the American Heart Association, New York City Affiliate, released Mar. 14, 1994.)

Route of Administration

The adenosine receptor antagonists described herein, e.g., 3-[4-(2,6-Dioxo-1,3-dipropyl-2,3,6,7-tetrahydro-1H-purin-8-yl)-bicyclo[2.2.2]oct-1-yl]-propionic acid, are generally administered to a subject parenterally. Parenteral formulations may be a single bolus dose, an infusion or a loading bolus dose followed with a maintenance dose. In some embodiments, the dosage (e.g., a single dosage form described herein) is administered by infusion. For example, a composition including an adenosine receptor antagonist such as 3-[4-(2,6-Dioxo-1,3-dipropyl-2,3,6,7-tetrahydro-1H-purin-8-yl)-bicyclo[2.2.2]oct-1-yl]-propionic acid is administered to a subject by infusing the subject with the composition including the adenosine receptor antagonist. The infusion generally spans from about 5 minutes to about 2 hours (e.g., about 10 minutes, about 15 minutes, about 20 minutes, about 30 minutes, about 45 minutes, about 1 hours, about 1.5 hours, or about 2 hours). In some embodiments, the subject is administered a 30 minute infusion.

The adenosine receptor antagonist is generally administered to the subject from about 1 times per day to about 4 times per day (e.g., 1, 2, 3, or 4 times per day). In some embodiments, the adenosine receptor antagonist such as 3-[4-(2,6-Dioxo-1,3-dipropyl-2,3,6,7-tetrahydro-1H-purin-8-yl)-bicyclo[2.2.2]oct-1-yl]-propionic acid is administered 2 times per day, for example, about every 12 hours.

In some embodiments, IV administration of an adenosine receptor antagonist occurs during the time of hospitalization of a subject. For example, IV administration occurs only while the subject is hospitalized. In some embodiments, a patient is selected for IV administration wherein the patient does not have an elevated risk of seizure or is at low risk of a seizure prior to administration of the adenosine receptor antagonist. For example, the subject does not have a history of past seizures (no struck or head trauma within the past six months, no seizure within the last ten years, and/or the subject has not been on any anti-seizure medication for the past five years. In some embodiments, upon selection of the patient, the patient is administered a compound or composition described herein. In some embodiments a patient is selected for IV administration such that, prior to administration, the patient has a glomerular filtration rate (GFR) of from about 10 to about 80, e.g., from about 20 to about 70.

Methods and Course of Treatment

The course of treatment of a subject can be one day or longer, e.g., 1, 2, 3, 4, 5, 6, 7, 8, 9, 10, 11, 12, 13, 14, 15, 16, 17, 18, 19, 20, 21 days, or more, e.g., 5 days. In some embodiments, a subject is treated with a course of treatment described herein, for example, 5 days of treatment with a parental formulation of an adenosine receptor antagonist such as 3-[4-(2,6-Dioxo-1,3-dipropyl-2,3,6,7-tetrahydro-1H-purin-8-yl)-bicyclo[2.2.2]oct-1-yl]-propionic acid (e.g., 2 times daily). Upon completion of the course of treatment, the subject can have an additional course of treatment, for example, and additional course of treatment such as described herein. In some embodiments, upon completion of a course of treatment with a parental formulation, the subject undergoes a subsequent course of treatment, for example with an oral formulation of an adenosine receptor antagonist such as 3-[4-(2,6-Dioxo-1,3-dipropyl-2,3,6,7-tetrahydro-1H-purin-8-yl)-bicyclo[2.2.2]oct-1-yl]-propionic acid (e.g., for at least one day, seven days, two weeks, three weeks, one month, or longer).

The compounds and compositions described herein can be administered to a subject, for example using a method described herein, who is suffering form a disorder where antagonizing the adenosine receptor would be beneficial. For example, adenosine receptor antagonists can be useful in the prevention and/or treatment of numerous diseases, including cardiac and circulatory disorders, degenerative disorders of the central nervous system, respiratory disorders, and many diseases for which diuretic treatment is suitable. In some embodiments, a compound or composition described herein, for example, a composition including 3-[4-(2,6-Dioxo-1,3-dipropyl-2,3,6,7-tetrahydro-1H-purin-8-yl)-bicyclo[2.2.2]oct-1-yl]-propionic acid is administered in the treatment of acute decompensated heart failure. In some embodiments, a compound or composition described herein, for example, a composition including 3-[4-(2,6-Dioxo-1,3-dipropyl-2,3,6,7-tetrahydro-1H-purin-8-yl)-bicyclo[2.2.2]oct-1-yl]-propionic acid is administered in a subject having concomitant renal insufficiency. In some embodiments, the subject is being treated for both acute decompensated heart failure and concomitant renal insufficiency.

In some embodiments, a compound or composition described herein can be used in the treatment of renal failure, for example, renal failure induced by a nephrotoxic agent such as cisplatin. In some embodiments, a compound or composition described herein can be used in the treatment of one or more other conditions associated with impaired renal function, for example, heart failure.

In some embodiments, adenosine receptor antagonist, e.g., in a composition described herein, is used to treat metabolic syndrome. Metabolic syndrome (e.g., Syndrome X) is characterized by a group of metabolic risk factors in one person. They include: central obesity (excessive fat tissue in and around the abdomen), atherogenic dyslipidemia (blood fat disorders—mainly high triglycerides and low HDL cholesterol—that foster plaque buildups in artery walls); insulin resistance or glucose intolerance (the body can't properly use insulin or blood sugar); prothrombotic state (e.g., high fibrinogen or plasminogen activator inhibitor [−1] in the blood); raised blood pressure (i.e., hypertension) (130/85 mmHg or higher); and proinflammatory state (e.g., elevated high-sensitivity C-reactive protein in the blood). The underlying causes of this syndrome include overweight/obesity, physical inactivity and genetic factors. People with metabolic syndrome are at increased risk of coronary heart disease, other diseases related to plaque buildups in artery walls (e.g., stroke and peripheral vascular disease) and type 2 diabetes. Metabolic syndrome is closely associated with a generalized metabolic disorder called insulin resistance, in which the body can't use insulin efficiently. In some embodiments, a compound or composition described herein can be used to treat type 2 diabetes.

In some embodiments, an adenosine receptor antagonist, e.g., in a composition described herein, is used to lower the level of triglycerides in a subject, for example, in the treatment of hyperlipidemia. Hyperlipidemia, which includes hyperlipoproteinemia, hypercholesterolemia, and hypertriglyceridemia, is characterized by elevated serum lipids. Lipids exist in the bloodstream (e.g., cholesterol, cholesterol esters, phospholipids, and triglycerides) as lipoproteins, which are associated with chylomicrons, very low-density lipoproteins (VLDL), intermediate-density lipoproteins (IDL), low-density lipoproteins (LDL), and high-density lipoproteins (HDL). Hyperlipidemia, or more specifically, hypercholesterolemia, is diagnosed in patients with total serum cholesterol greater than 200 mg/dl, or LDL levels greater than 130 mg/dl. Hyperlipidemia causes hardening of arteries, or atherosclerosis; increasing the risk of heart disease, stroke, and other vascular disease.

In some embodiments, the subject is being treated with an additional therapeutic agent, for example a diuretic such as a loop diuretic, an anti-seizure medication, an ACE inhibitor, an angiotensin receptor blocker, digoxin, and/or a beta blocker. Exemplary loop diuretics include furosemide, bumetanide, ethacrynic adic, and orsemide. Exemplary anti-seizure medications include cabamazepine, clobazam, diastat, ethosuximide, felbamate, frisium, gabapentin, klonopin, lamotrigine, levetiracetam, lorazepam, luminal, lyrica, oxcarbazepine, phenobarbital, phenytoin, primidone, rufinamide, sabril, tiagabine, topiramate, valproic acid, valproate, and zonisamide. Exemplary ACE inhibitors include captopril, zofenopril, enalapril, ramipril, quinapril, perindopril, lisinopril, and benazepril. Exemplary angiotensin receptor blockers include valsartan, telmisartan, losartan, irbesartan, and olmesartan. Exemplary beta blockers include alprenolol, carteolol, levobunolol, mepindolol, metipranolol, nadolol, oxprenolol, penbutolol, pindolol, propenolol, sotalol, timolol, acebutolol, atenolol, beaxolol, bisoprolol, esmolol, metoprolol, nebivolol, amosulalol, landiolol, tilisolol, arotinolol, carvedilol, celiprolol, and labetalol.

Clinical Outcomes

In some embodiments, treatment with a compound or composition described herein, for example, using a method described herein, improves one or more clinical outcomes. For example, in some embodiments, treatment with a compound or composition described herein reduces mortality and/or reduces re-hospitalization (e.g, increases the amount of time to mortality and/or re-hospitalization by at least 1 day, by at least 2 days, by at least 3 days, by at least one week, by at least 2 weeks, by at least 3 weeks, by at least one month, etc.) relative to a subject not treated with a compound or composition described herein (e.g., a composition including 3-[4-(2,6-Dioxo-1,3-dipropyl-2,3,6,7-tetrahydro-1H-purin-8-yl)-bicyclo[2.2.2]oct-1-yl]-propionic acid.). In some embodiments, treatment with a compound or composition described herein increases the time to mortality and/or re-hospitalization by at least 5%, 10%, 15% or more relative to a subject not treated with a compound or composition described herein (e.g., a composition including 3-[4-(2,6-Dioxo-1,3-dipropyl-2,3,6,7-tetrahydro-1H-purin-8-yl)-bicyclo[2.2.2]oct-1-yl]-propionic acid.).

In some embodiments, treatment with a compound or composition described herein, for example using a method described herein, results in the promotion of natriuresis, reduction of body weight, and/or preservation of renal function in a subject, (e.g., a subject having stable heart failure). Analysis of one or more clinical outcomes can be evaluated using methods known in the art, for example, evaluation of safety assessments, pharmakinetic parameters, eGFR, weight change, sodium excretion, urine volume, adjusted creatine clearance (e.g., a reduction in accumulation of serum creatinine, for example, by at least about 10%, at least about 20%, at least about 320%, at least about 40%, at least about 50%, or t least about 60%), natriuesis, and hemodynamic change. For example, in some embodiments, a weight loss off about 0.5 kg or more (e.g., 0.6 kg) can be observed upon treatment with a compound or composition described herein, e.g., a composition including 3-[4-(2,6-Dioxo-1,3-dipropyl-2,3,6,7-tetrahydro-1H-purin-8-yl)-bicyclo[2.2.2]oct-1-yl]-propionic acid. In some embodiments, a subject will have an improvement of from about 5% to about 10% in weight loss where the patient has also shown an improvement in dyspnea score, relative to the weight loss in a patient that does not show an improvement in dyspnea score.

In some embodiments, treatment with a compound or composition described herein, for example using a method described herein, results in an improved score for the subject in the NYHA (New York Heart Association) classification system (e.g., improvement in a subject having a score of III or IV prior to treatment with a compound or composition described herein). This improvement can be measured in a variety of manners such as the ability to participate in physical activity (e.g., measurement of exercise capacity).

In some embodiments, treatment with a compound or composition described herein can result in decreased cardiac vasculitis, decreased degenerative ischemic changes, and/or decreased degenerative necrosis. In some embodiments, treatment with a compound or composition described herein can result in improved pericarditis and/or aortic vasculitis.

In some embodiments, treatment with a compound or composition described herein can result in attenuated progression of diabetic nephropathy. In some embodiments, treatment with a compound or composition described herein can result in attenuated progression of cardiac histopathological changes, for example, those associated with dilated cardiomyopathy and/or diabetes.

In some embodiments, a subject is monitored for improvement in one or more conditions or parameters described herein. For example, a subject is monitored subsequent to treatment with an receptor antagonist, such as a compound described herein, for example administered by a method described herein. The subject can be evaluated, for example, by assessment of one or more pharmacodynamic parameters. Exemplary pharmacodynamic parameters include sodium excretion, urine volume, adjusted creatinine clearance, hemodynamics, and body weight.

In some embodiments, a subject is monitored for one or more adverse events. Adverse events can be a treatment related event or a non-treatment related event. Exemplary adverse events include seizure (e.g., status epilepticus), an ischemic event such as a transient ischemic attach and headache.

### EXAMPLES

### Example 1

### Liquid dosage form of 3-[4-(2,6-Dioxo-1,3-dipropyl-2,3,6,7-tetrahydro-1H-purin-8-yl)-bicyclo[2.2.2]oct-1-yl]-propionic acid

A liquid dosage form of 3-[4-(2,6-Dioxo-1,3-dipropyl-2,3,6,7-tetrahydro-1H-purin-8-yl)-bicyclo[2.2.2]oct-1-yl]-propionic acid includes 20 mg/Ml of 3-[4-(2,6-Dioxo-1,3-dipropyl-2,3,6,7-tetrahydro-1H-purin-8-yl)-bicyclo[2.2.2]oct-1-yl]-propionic acid, 25 mM of histidine buffer, and 3.25% mannitol.

The phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of administering an adenosine receptor antagonist, the method comprising administering to a subject in need thereof a liquid formulation comprising the adenosine receptor antagonist by infusing the liquid formulation into the subject, wherein the amount of adenosine receptor antagonist administered to the subject is from about 0.03 mg/kg to about 3.0 mg/kg.

2. The method of claim 1, further comprising reconstituting a solid formulation comprising the adenosine receptor antagonist to produce the liquid formulation.

3. The method of claim 1, wherein the amount of adenosine receptor antagonist administered to the subject is from about 0.03 mg/kg to about 1.0 mg/kg.

4. The method of claim 3, wherein the amount of adenosine receptor antagonist administered to the subject is from about 0.03 mg/kg to about 0.3 mg/kg.

5. The method of claim 1, wherein the adenosine receptor antagonist is infused over a course of about 30 minutes.

6. The method of claim 1, wherein administering the adenosine receptor antagonist promotes natriuresis, reduces body weight or preserves renal function.

7. The method of claim 1, wherein the adenosine receptor antagonist is administered once or twice daily.

8. The method of claim 1, wherein the total daily dose of the adenosine receptor antagonist is from about 6 mg to about 50 mg.

9. The method of claim 1, wherein the liquid formulation further comprises histidine.

10. The method of claim 1, wherein the pH of the liquid formulation is at least about 8.0.

11. The method of claim 1, wherein the liquid formulation further comprises a base.

12. The method of claim 1, further comprising a pharmaceutically acceptable carrier.

13. The method of claim 1, wherein the adenosine receptor antagonist is 3-[4-(2,6-Dioxo-1,3-dipropyl-2,3,6,7-tetrahydro-1H-purin-8-yl)-bicyclo[2.2.2]oct-1-yl]-propionic acid.

14. The method of claim 1, wherein the adenosine receptor antagonist is administered in combination with at least one other therapeutic agent.

15. The method of claim 14, wherein the other therapeutic agent is an anti-seizure medication, a diuretic, an ACE inhibitor, an angiotensin receptor blocker, or a beta blocker.

16. The method of claim 1, wherein the subject is suffering from acute decompensated heart failure.

17. The method of claim 16, wherein the subject is also suffering from concomitant renal insufficiency.

18. The method of claim 1, wherein said subject is at low risk for seizures.

19. The method of claim 1, wherein said subject has a GFR of 10-80.

20. The method of claim 1, wherein the adenosine receptor antagonist is administered to a subject wherein an ischemic event is imminent.

21. The method of claim 1, wherein the adenosine receptor antagonist is administered to a subject within two days after an ischemic event.

22. The method of claim 1, further comprising monitoring the subject for an adverse event.

23. The method of claim 1 wherein the subject is a mammal.

24. The method of claim 23 wherein the mammal is a human.
